(19) United States
(12) Patent Application Publication
(10) Pub. No.: US 2026/0076922 A1
(43) Pub. Date: Mar. 19, 2026

(54) DECREASING ACINETOBACTER POPULATIONS IN THE MICROBIOME

(71) Applicant: GLYCIOME, LLC, Valleyford, WA (US)

(72) Inventors: Joanna E. Ellington, Valleyford, WA (US); G. D. Clifton, Valleyford, WA (US); Rayne Ellington-Lawrence, Valleyford, WA (US)

(21) Appl. No.: 19/401,625

(22) Filed: Nov. 26, 2025

Related U.S. Application Data
(63) Continuation of application No. PCT/US2024/031587, filed on May 30, 2024.
(60) Provisional application No. 63/505,325, filed on May 31, 2023.

(51) Int. Cl. A61K 31/045; A61K 31/22; A61K 31/7016; A61K 33/32; A61P 15/00

(52) U.S. Cl. CPC A61K 31/045; A61K 31/22; A61K 31/7016; A61K 33/32; A61P 15/00

(57) ABSTRACT

The present disclosure provides compositions and methods for compositions for use in optimizing the genital microbiome of a user or sexual partners of that user. The compositions may comprise a prebiotic oligosaccharide, a metal co-factor, and an essential oil comprising bornyl acetate. The compositions support the genital microbiota and are useful for, for example, decreasing the growth of pathogenic bacteria such as bacteria from the species Acinetobacter. Furthermore, the compositions may have minimal or beneficial effect on gametes.

### CROSS-REFERENCE TO RELATED APPLICATIONS

This present application is a continuation application of International Patent Application No. PCT/US2024/031587, filed May 30, 2024, which claims priority to U.S. Provisional Patent Application Ser. No. 63/505,325, filed May 31, 2023, which are herein incorporated by reference in their entirety.

### BACKGROUND

The genital microbiome of a human is a unique combination of microbial species comprising at least one hundred species of bacteria and a variety of fungal, viral, and protozoal species. There is considerable variation in make-up of the genital microbiome between individuals, with many factors such as hygiene regimes, diet, environment, age, ethnicity, disease, sexual activity, sexual orientation, and life history affecting the presence of specific microbial species and their metabolic activities. Environmental conditions within the genitalia compared to other locations of the human body are distinct. As a result, products indicated for urogenital, anogenital, vaginal and/or penile use, to modulate the genital microbiome, may require distinct prebiotic and/or probiotic constituents.

The genital dysbiosis (e.g., penile growth dysbiosis, vaginal dysbiosis) may be etiologically connected with various sexually transmitted diseases such as HIV, HSV, or HPV, urethritis, abnormal sperm quality, and penile cancers.

One particular genus present in the microbiome, species from the genus Acinetobacter such as Acinetobacter baumannii, have been shown in other media to be correlated with severe infections. Acinetobacter infections are often nonsocomial infections resulting in severe sepsis or severe shock following exposure in many intensive care units in hospitals (ICUs). Acinetobacter species are often characterized by a great persistence in the environment enabling them to spread rapidly and have the ability to develop resistance to conventional antimicrobials and biocides.

Products often administered to these regions typically lead to a variety of bothersome symptoms and pathologies from odor to itching, inflammation, biofilm production (which may protect pathogens from the host immune system), mucin secretion degradation, and higher risks of viral infections and cancer. Certain life events cause an individual to be more susceptible to dysbiosis, including: ovulation, menses, birth, puberty, pregnancy, sexual initiation, non-circumcision of the male, genital mutilation of the female, diseases (e.g., diabetes), introduction of new sexual partners, post-partum, involution of the uterus, times of immunosuppression, menopause or andropause, and cancer therapy.

There remains a need for compositions for use in the urogenital and/or anogenital region that are not harmful to the genital tissues and that preserve and support the beneficial regional microbiome of these areas while decreasing incidence of Acinetobacter populations therein. Presently disclosed embodiments address this need and provide other related advantages.

### SUMMARY

The present disclosure provides compositions and methods for maintaining and/or optimizing the microbiome of certain subject bodily regions including the urogenital (e.g., subject regions relating to function of urinary excretion and reproduction) and/or anogenital regions (e.g., relating to the anus and genitals) by application of a composition capable of reducing Acinetobacter species present in the microbiome. These methods are partially based on the discovery that compositions designed to increase the beneficial microbiota of the microbiome also decrease the population of Acinetobacter species present therein. In particular, the present disclosure relates to topical compositions for administration to the urogenital (e.g., subject regions relating to function of urinary excretion and reproduction) and/or anogenital regions (e.g., relating to the anus and genitals) of a subject in order to decrease the Acinetobacter population in the subject. These compositions may comprise a prebiotic oligosaccharide, a metal co-factor, and borneol, or pharmaceutically acceptable salts or prodrugs thereof such as bornyl acetate (e.g., via an essential oil comprising bornyl acetate), and may have an acidic pH level (e.g., less than 7 or less than 6.5 or less than 6 or less than 5.5 or less than 5 or from 4 to 6 or from 4 to 5 or from 4.25 to 4.75). The compositions of the present disclosure may be used for treatment or prophylaxis of urogenital infection, decreasing penile cancer persistence or occurrence, improving penile cancer treatment, or preventing penile cancer occurrence. Furthermore, these methods may also decrease the occurrence of urogenital infection and or cancer of the genitals in a sexual partner of the subject having applied the compositions disclosed herein.

In some embodiments, the compositions (e.g., topical, topical isotonic, gels, lubricants) of the present disclosure comprise:
(a) a metallic co-factor (e.g., manganese chloride);
(b) a prebiotic oligosaccharide (e.g., lactulose); and
(c) borneol or a prodrug thereof (e.g., bornyl acetate);
   wherein the composition is optionally buffered with a buffer system comprising gluconolactone. In particular, the compositions provide an increased buffering capacity in a pH range to support beneficial microbiome optimization for a person in need thereof (e.g., in the vaginal microbiome). The compositions may, for example, have increased buffering capacity in a pH range of from 4-7 or from 5-7 or from 5-6.

### BRIEF DESCRIPTION OF THE FIGURES

FIG. 1 shows the relative abundance of Acinetobacter and Veillonella in the microbiome following baseline (left), Astroglide® (middle), and composition of the present disclosure (right, BioMa) test conditions to three healthy males. Error bars illustrate standard deviation.

### DETAILED DESCRIPTION

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. Any concentration ranges recited herein are to be understood to include concentrations of any integer within the range and fractions thereof, such as one tenth, one hundredth, and one thousandth of an integer, unless otherwise indicated. Unless otherwise indicated, it will be understood that any percentage refers to the weight percentage with respect to the indicated component. Typically, the percent of a component in the composition indicates the weight percentage with respect to the weight of the composition.

The term “consisting essentially of” is not equivalent to “comprising” and refers to the specified materials or steps, or to those that do not materially affect the basic and novel characteristics of the claimed subject matter. It should be understood that the terms “a” and “an” as used herein refer to “one or more” of the enumerated components.

A dyad is often a group of two persons having a sociologically significant relationship (e.g., sexual relationship, parent-infant relationship, parent-child relationship, health care provider and patient relationship, care-giver and patient relationship). Application of the compositions described herein may optimize or maintain the microbiome of one or more members of a sexual relationship including a sexual dyad. The dyad may be composed of two males, one male and one female, or other gender groupings (e.g. non-binary gendered individual, intersex individual). In certain embodiments, a dyad may be a sexual dyad such heterosexual dyad, a homosexual dyad, or other sexual orientation dyad. In other embodiments, the sexual relationship may be non-binary such as a sexual triad (a group of three people).

The genital microbiota or genital microbiome may include the collective microorganisms that normally colonize the genital region. The genital microbiota may be non-pathogenic. The genital microbiota may refer to that of the genital skin microbiota, the vaginal microbiota (e.g., vaginal mucosal microbiota) of a female subject, the cervical microbiota of a female subject, penile microbiota (e.g., penile skin microbiota such as the foreskin microbiota or the urethral meatus microbiota) of a male subject, microbiota of the genital tissue of an intersex individual, microbiota of a non-binary gendered individual, or any combination thereof. Recently, species overlap between rectal and urinary microbiome species in an individual have been observed. For example, the bacteria of the genital region have been found to represent a continuum between organs of excretion and reproduction as discussed Y. Govender, et al., Front Cell Infect Microbiol 9 (2019): 133, hereby incorporated by reference in its entirety and particularly in relation to the urinary microbiomes disclosed therein. These bacteria of the genital region are referred to herein as the anogenital and/or urogenital microbiomes.

Genital probiotic bacteria may refer to live bacteria, which when administered in adequate amounts to the vagina or penis confer a health benefit (e.g., such as those described herein) to the host subject.

The vaginal microbiota is often affected by the penile microbiota and correlations between BV and penile microbiota between members of a sexual dyad have been shown in C. Liu, et al., mBio 6 (2015): e00589-15, hereby incorporated by reference in its entirety and particularly in relation to the exchange of BV associated bacteria through intercourse and connections between Nugent score and penile community state types. Penile microbiota or penile flora may refer to the collective microorganisms that normally colonize the penis, foreskin, and distal urethra which are non-pathogenic. The penis includes the penile shaft and distal glans, which includes the glans, glans coronal, meatus urethralis, fossa navicularis, frenulum, coronal sulcus, and foreskin. In various implementations, the penile microbiota, and in particular, in an optimized penile microbiome, comprises bacterial species from the genus Lactobacillus, Streptococcus, Staphylococcus, Corynebacteria, and combinations thereof. In certain embodiments, the penile microbiota comprises Lactobacillus crispatus, Lactobacillus jensenii, Lactobacillus iners, Lactobacillus gasseri, Streptococcus, non-pathogenic Prevotella, Corynebacteria, Staphylococcus, Anaerococcus, Peptoniphilus, Finegoldia, Porphyromonas, Propionibacterium, Delftia, Bifidobacterium, Clostridium, non-pathogenic Pseudomonas, or any combination thereof. In certain embodiments, the penile microbiota of a male subject reflects the vaginal microbiota and/or reproductive tract microbiota of a female subject, wherein the male subject and female subject are members of a sexual dyad. The species found in normal penile microbiota can differ between circumcised and uncircumcised subjects. For example, penile microbiota may also include sperm microbiota such as those disclosed in D Baud, et al., Frontiers in Microbiol 10 (2019): 234, hereby incorporated by reference in its entirety and particularly in relation to beneficial seminal microbiota including Lactobacillus species. In some embodiments, the compositions are able to increase the number of beneficial species in the penile microbiome (and by extension the vaginal microbiome following intercourse) such as Lactobacillus, Streptococcus, Staphylococcus, Corynebacteria, and optimize or balance (e.g., decrease and/or increase) the number of dysbiosis associated anaerobes such as those from the species Prevotella, Finegoldia, Diallister, Snethia, Megasphaeae, Mobiluncus, Mycoplasma, Peptococcus, Peptostreptococcus, Porphyromonas, Slackia, Tannerella, Treponema, Ureaplasma, Veillonella, Acinetobacter, Anaerococcus, Actinomyces, Aggregatibacter, Atopobium, Bacteroides, Bifidobacteriium, Clostridiales, Eggerthella, Eubacterium, Fusobacterium, Garnderella, Leptotrichia, and combinations thereof. In some embodiments, optimization of the microbiome involves decreasing the number of pathogenic communities of bacteria such as those species from the genera Gardnerella, Finegoldia, Dialister, Prevotella, Anaerococcus, Atopobium, Acinetobacter, Megasphaera, and combinations thereof. In particular, the present disclosure relates to decreasing the population of Acinetobacter species present in the microbiome such as one or more of Acinetobacter albensis, Acinetobacter apis, Acinetobacter baumannii, Acinetobacter baylyi, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter celticus, Acinetobacter chengduensis, Acinetobacter colistiniresistens, Acinetobacter courvalinii, Acinetobacter cumulans, Acinetobacter defluvii, Acinetobacter dispersus, Acinetobacter dijkshoorniae, Acinetobacter equi, Acinetobacter gandensis, Acinetobacter gerneri, Acinetobacter guangdongensis, Acinetobacter guerrae, Acinetobacter guillouiae, Acinetobacter gyllenbergii, Acinetobacter haemolyticus, Acinetobacter harbinensis, Acinetobacter indicus, Acinetobacter junii, Acinetobacter kookii, Acinetobacter lactucae, Acinetobacter lanii, Acinetobacter larvae, Acinetobacter lwoffii, Acinetobacter modestus, Acinetobacter nectaris, Acinetobacter nosocomialis, Acinetobacter oryzae, Acinetobacter parvus, Acinetobacter pakistanensis, Acinetobacter populi, Acinetobacter portensis, Acinetobacter proteolyticus, Acinetobacter pittii, Acinetobacter piscicola, Acinetobacter pragensis, Acinetobacter proteolyticus, Acinetobacter pseudolwoffii, Acinetobacter pullicarnis, Acinetobacter pullorum, Acinetobacter puyangensis, Acinetobacter qingfengensis, Acinetobacter radioresistens, Acinetobacter rudis, Acinetobacter schindleri, Acinetobacter seifertii, Acinetobacter shaoyimingii, Acinetobacter soli, Acinetobacter stercoris, Acinetobacter tandoii, Acinetobacter tjernbergiae, Acinetobacter towneri, Acinetobacter ursingii, Acinetobacter variabilis, Acinetobacter venetianus, Acinetobacter vivianii, Acinetobacter wanghuae, Acinetobacter wuhouensis, and combinations thereof. For example, the compositions of the present disclosure may decrease the population of Acientobacter baumannii in the microbiome of a subject which may provide therapeutic benefit.

The anogenital region of a subject includes regions of the anus and the genitalia. In certain embodiments, the female anogenital region comprises the cervix, vagina, vulva, clitoris, urethral meatus, urethral meatus, vulval vestibule, perineum, and/or anus. In certain embodiments, the male anogenital region comprises the penis, base of the penis, foreskin, urethral meatus, scrotum, perineum, and anus. The term urogenital region may refer to the region of the distal urinary tract and the genitalia. In certain embodiments, the female urogenital region comprises the cervix, vagina, vulva, clitoris, introitus, urethral meatus, urethral fold, vulval vestibule, and/or perineum. In some subjects, the anogenital and/or urogenital regions of a subject may be indistinct, intersex, or transitioning from male to female or female to male due to iatrogenic (e.g., surgery or hormone therapy) or natural/genetic causes.

Genital tissues are often living cells found in the anogenital and/or urogenital regions. Genital tissues include, but are not limited to epithelial surface cells (e.g., skin), mucosal cells, immune cells, nerve cells, blood cells, connective tissue cells, and neoplastic cells of the vulva, clitoris, vagina, vestibule, vulval vestibule, urethral meatus, penis, foreskin, distal urethra, and scrotum. Since sperm cells exit the penis and are often deposited on genital tissue (e.g., vagina), genital tissues also include semen and sperm cells.

“Effective amount” or “therapeutically effective amount” refers to that amount of a composition of this disclosure which, when administered to a subject, such as a human, is sufficient to affect a desired biological effect or treatment including optimizing of the penile microbiome or decreasing the population of Acinetobacter presence therein. In some embodiments, the effective amount may have minimal effect on gametes following application (e.g., with minimal changes in motility, concentration, vitality, morphology of gametes, oxidation-reduction potential, sperm DNA fragmentation, sperm mitochondrial membrane potential, survival, and changes at the sub-cellular levels such as changes to proteins related to specific functions of the gametes). In some embodiments, the therapeutically effective amount alters one or more features of gametes in the genital fluids of the user by less than 20%.

As used herein, the term “subject” refers to any organism to which a composition in accordance with the disclosure may be administered, e.g., for experimental, diagnostic, prophylactic, and/or therapeutic purposes. Typical subjects include any animal (e.g., mammals such as mice, rats, rabbits, non-human primates, and humans, etc.). A subject in need thereof is typically a subject for whom it is desirable to treat a disease, disorder, or condition as described herein (and in particular, treatment of a disease, disorder or condition relating to dysbiosis of the urogenital and/or anogenital regions). For example, a subject in need thereof may seek or be in need of treatment, require treatment, be receiving treatment, may be receiving treatment in the future, or a human or animal that is under care by a trained professional for a particular disease, disorder, or condition.

As used herein, the phrase “pharmaceutically acceptable” generally safe for ingestion or contact with biologic tissues at the levels employed. Pharmaceutically acceptable is used interchangeably with physiologically compatible.

The compounds described herein may be present as a pharmaceutically acceptable salt. Typically, salts are composed of a related number of cations and anions (at least one of which is formed from the compounds described herein) coupled together (e.g., the pairs may be bonded ionically) such that the salt is electrically neutral. Pharmaceutically acceptable salts may retain or have similar activity to the parent compound (e.g., an ED₅₀ within 10%) and have a toxicity profile within a range that affords utility in pharmaceutical compositions. For example, pharmaceutically acceptable salts may be suitable for use in contact with the tissues of humans and animals without undue toxicity, irritation, allergic response and are commensurate with a reasonable benefit/risk ratio. Pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, dichloroacetate, digluconate, dodecylsulfate, ethanesulfonate, formate, fumarate, glucoheptonate, glutamate, glycerophosphate, hemisulfate, heptonate, hexanoate, hippurate, hydrobromide, hydrochloride, hydroiodide, 2-hydroxy-ethanesulfonate, isethionate, lactobionate, lactate, laurate, lauryl sulfate, malate, maleate, malonate, mandelate, methanesulfonate, mucate, 2-naphthalenesulfonate, nicotinate, nitrate, oleate, oxalate, palmitate, pamoate, pantothenate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative basic salts include alkali or alkaline earth metal salts include sodium, lithium, potassium, calcium, and magnesium, aluminum salts, as well as nontoxic ammonium, quaternary ammonium, and amine cations, including, but not limited to ammonium, tetramethylammonium, tetraethylammonium, methylamine, dimethylamine, trimethylamine, triethylamine, caffeine, and ethylamine.

Pharmaceutically acceptable acid addition salts of the disclosure can be formed by the reaction of a compound of the disclosure with an equimolar or excess amount of acid. Alternatively, hemi-salts can be formed by the reaction of a compound of the disclosure with the desired acid in a 2:1 ratio, compound to acid. The reactants are generally combined in a mutual solvent such as diethyl ether, tetrahydrofuran, methanol, ethanol, iso-propanol, benzene, or the like. The salts normally precipitate out of solution within, e.g., one hour to ten days and can be isolated by filtration or other conventional methods.

Prodrugs are typically compounds that may be converted under physiological conditions or by solvolysis to a biologically active compound of the invention. Prodrug may refer to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the indicated compound, for example, by hydrolysis in blood. The prodrug compound often offers advantages of solubility, tissue compatibility or delayed release in a mammalian organism as described in Bundgard, H., Design of Prodrugs (1985): 7-9, 21-24 (Elsevier, Amsterdam), Higuchi, T., et al., ACS Symposium Series, Vol. 14, and Bioreversible Carriers in Drug Design, Ed. Edward B. Roche, American Pharmaceutical Association and Pergamon Press, 1987, each of which are hereby incorporated by reference in their entirety.

The present disclosure provides compositions (e.g., acidic compositions, topical isotonic compositions, acidic topical isotonic compositions) which may comprise a prebiotic oligosaccharide, a metal co-factor, and bornyl acetate (e.g., the composition comprises an essential oil comprising bornyl acetate). Such compositions have no or minimal irritation to the epithelial tissues of the urogenital and/or anogenital regions; no or minimal disruption of natural mucus secretions and mucins of the urogenital and/or anogenital regions; minimal or no inhibition on the normal, non-pathogenic microbiota of the urogenital and/or anogenital regions; do not promote growth of pathogenic bacteria of the urogenital and/or anogenital regions (or decrease their populations such as populations of Acinetobacter species); limit transfer of pathogenic bacteria from a first member of a dyad to a second member of a dyad; enhance transfer of beneficial microbiota species from a first member of a dyad to a second member of a dyad; or any combination thereof.

It is important to consider the genital microbiome and its implications on human health in the development of genital and sexual health products and therapeutics. In general, the healthy human vaginal microbiome, which is dominated by predominantly Lactobacilli species, maintains and utilizes a carbohydrate-based, antioxidant-rich environment (Chen et al. Nat Commun 8:875, 2017, hereby incorporated by reference in its entirety). These Lactobacilli produce distinct metabolites that stimulate epithelial mucus production, regulate immune cell reactivity, control pathogenic biofilm and biogenic amine formation, and prevent the colonization of the vaginal tract by pathogenic bacteria. The penile microbiome may reflect, and often is correlated to, the vaginal microbiome of a female sexual partner. Dysbiosis, which refers to impaired or imbalanced microbiota, can lead to outgrowth of pathogenic, or dysbiotic, microorganisms. Dysbiotic organisms tend to prefer amino acids or proteins as energy substrates rather than carbohydrates, produce an antioxidant-poor environment (which is high in cytokine active metabolites) degradation products such as biogenic amines, and certain antioxidant reducing agents. Without wishing to be bound by theory, Acinetobacter species in particular target multiple O-linked host glycoproteins to gain dominance over beneficial microbiota to become associated with consequential urogenital infections, and, preliminarily, with penile cancer.

In some embodiments, the compositions of the present disclosure may be administered to the anogenital and/or urogenital region of a male. In some embodiments, the compositions may be administered to the anogenital and/or urogenital region of a female, and particularly, a pregnant female. Furthermore, application of the compositions of the present disclosure may have minimal (e.g., within 20% of one or more measured parameters such as motility) if not beneficial, effect on gametes (e.g., male gametes, female gametes). In some embodiments, application of the compositions of the present disclosure may have minimal (e.g., within 20% of one or more measured parameters such as motility) if not beneficial, effect on zygotes.

Additionally, urinary incontinence, diabetes, genital lichen sclerosus, interstitial cystitis, and autoimmune conditions can increase genital dysbiosis (Thomson, J. Reprod. Med. 50:513, 2005; Alam et al. Immune Netw 14:7, 2014). Individuals with such diseases can be very sensitive to disease exacerbation via ingredients or chemicals in formulations currently used for routine genital care and intimacy as disclosed in A Chung, et al., World J Urol (2019): 1-5.

Genital dysbiosis is also common in individuals undergoing gender reassignment surgery. Active and ongoing management of newly created genitalia requires daily or as needed cleaning and intervention to assist in healthy genital biome development of these newly created regions (see, Weyers et al., BMC Microbiol. 9:102, 2009). Furthermore, with increased prevalence of Actineobacter nosocomial infections, these patient populations may be particularly susceptible to Actineobacter infection. In various embodiments, the subject in need thereof is an individual undergoing or having undergone gender reassignment surgery.

In some embodiments, subjects in need thereof may include having any one of the indications as described herein. The compositions of the present disclosure may be administered to the subject in need thereof for the treatment or prophylaxis of such conditions often related to dysbiosis. Additionally, the compositions of the present disclosure may be administered to the sexual partner of the subject in need thereof for the treatment or prophylaxis of such conditions often related to dysbiosis.

Overall, these diseases, disorders, and/or conditions are poorly controlled and poorly managed in many populations. Furthermore, the etiology of certain systemic disease conditions such as cancer (e.g., penile cancer) are connected with dysbiosis of the anogenital and/or urogenital regions. In some embodiments, the methods of the present disclosure may include a diagnosis from a medical professional of penile cancer and/or urogenital infection from a species of the genus Acinetobacter such as Acinetobacter baumannii to the subject or a member of the sexual relationship.

### Methods of Use

In one aspect, the present disclosure provides a method of decreasing the Acinetobacter population in the microbiome of the urogenital and/or anogenital region of a subject comprising topically administering an effective amount of a topical, isotonic composition of the present disclosure to the urogenital and/or anogenital region of the subject. Administration of the topical, isotonic composition of the disclosure may occur as needed such as one or more times daily, twice a week, three times a week, weekly, biweekly (e.g., every two weeks), monthly, bimonthly (e.g., every two months), etc. The method may be used to hydrate or moisturize the genital tissues, including the perineum, penis, or vulva/vagina. Irritation of the skin of the penis and disruption of healthy mucin secretions that moisten and protect the penis can increase dryness, roughness and inflammation resulting in pain and discomfort. Vaginal intercourse, which introduces the penile surface to a very low pH environment can irritate the skin of the penis. Similarly, irritation of the vagina skin around the vulva can occur following ejaculation of high pH and hypertonic semen into/onto the female genital region, leading to burning and post-coital pain. Continuous washing and cleaning of the urogenital and/or anogenital region, particularly with alkaline soaps or washes, can also irritate and dry out the urogenital and/or anogenital region. By hydrating or moisturizing the urogenital and/or anogenital region and buffering pH the topical, isotonic compositions of the present disclosure may be used to promote, enhance, protect the anogenital epithelium and microbiota. In certain embodiments, topical, isotonic compositions further comprise an additional therapeutic agent, such as a topical pain-relieving agent.

Methods are provided for the prophylaxis of cancer comprising administering the compounds of the present disclosure to the urogenital and/or anogenital region of a subject in need thereof (e.g., a subject having a dysbiotic microbiome with an increased population of Acinetobacter species as compared to a healthy microbiome). For example, the method may comprise administration of a composition of the present disclosure such as a composition comprising a metallic cofactor, a prebiotic oligosaccharide, and borneol or a prodrug thereof (e.g., bornyl acetate). In particular embodiments, the composition is administered to the anogenital and/or urogenital region (e.g., penis, vagina). of the subject in need thereof. Such administration may decrease prevlance of Acinetobacter populations in the microbiota which may lead to oncogenesis of these various cancerous cells.

Administration of the compositions of the present disclosure may selectively support epithelial colonization by Lactobacillus species such as L. crispatus, L. jensenii, and L. gasseri. In some embodiments, administration may suppress pathobionts linked to cancers such as penile cancer. In certain implementations, administration may maintain a constant or nearly constant non-inflammatory mucosal environment in comparison to baseline (no product use).

Administration of the topical, isotonic composition of the disclosure may occur as needed, daily, twice daily, three times a day, twice a week, three times a week, weekly, biweekly (e.g., every two weeks), monthly, bimonthly (e.g., every two months), etc. The topical, isotonic compositions of the present disclosure may be used for genital skin and mucosa conditioning and genital microbiome support, particularly in an aging subject (e.g., 50 years or greater, 60 years or greater, 70 years or greater, 80 years or greater, 90 years or greater, 100 years or greater) or a subject experiencing menopause or andropause. In certain embodiments, the topical, isotonic composition further comprises an additional therapeutic agent, such as a hormone, erectile dysfunction treatment or erectile enhancement drug, premature ejaculation drug, or a combination thereof.

In another aspect, the present disclosure provides a method of lubricating the urogenital and/or anogenital region of a subject comprising topically administering an effective amount of a composition of the present disclosure to the urogenital and/or urogenital and/or anogenital region of the subject. Administration of the composition of the disclosure may occur prior to, during, and/or after sexual intercourse or sexual activity, as needed such as one or more times daily, twice daily, three times a day, twice a week, three times a week, weekly, biweekly (e.g., every two weeks), monthly, bimonthly (e.g., every two months), etc. In certain embodiments, the urogenital and/or anogenital region is the perineum, vagina, vulva, clitoris, penis, scrotum, or anus. The topical composition may be administered to the urogenital and/or anogenital region of the subject prior to, during, and/or after sexual activity. Sexual activity includes oral sex, penetrative sex (e.g., vaginal intercourse, anal intercourse), non-penetrative sex, genital contact with a body part (e.g., hand, foot), genital contact with an object (e.g., sex toy), masturbation, dry humping (genital rubbing), or any combination thereof. In certain embodiments, the composition is administered to the urogenital and/or anogenital region of the subject for use with a sex toy. In additional embodiments, the composition is administered to a medical device, contraceptive device, or sex toy in alternative or in addition to administration to the urogenital and/or anogenital region. For example, the composition may be administered to the interior or exterior of a condom, to the interior or exterior of a sex toy, to the exterior or interior of a tampon, to the exterior of a menstrual cup, to the exterior of a diaphragm, or to the exterior of a vaginal ultrasound or speculum prior to contact with the subject's urogenital and/or anogenital region.

In yet another aspect, the present disclosure provides a method of supporting, enhancing, or promoting the genital microbiota of a subject or a sexual partner of a subject comprising topically administering an effective amount of a composition of the present disclosure to the genital region of the subject. In certain embodiments, the topical, isotonic composition is prebiotic for Lactobacillus species growth. In certain embodiments, the topical, isotonic composition may further comprise at least one probiotic bacterial species (e.g., Lactobacillus species). In various implementations, the composition of the present disclosure decreases Acinetobacter populations in the anogenital or urogenital region of the subject.

These compositions may be used for cleansing, and as a leave-in conditioner to enhance the healthy genital microbiome, and as a coital lubricant applied to the vaginal canal prior to intercourse or sexual activity. These compositions have a tonicity of 240 mOsmo/kg and a pH of from pH 3.5 to pH 6.8 (e.g., 4-6). Furthermore, the acidic compositions of the present disclosure may have minimal effect (e.g., more than 20% of one or more measured parameters such as sperm motility) on male gametes, female gametes, and/or zygotes.

In certain embodiments, the pH of the composition is formulated at a pH to match to the normal, physiological genital fluid pH (e.g., CVF, urethral secretions, semen) or at a pH appropriate for the particular method of use. In certain embodiments, the pH of the topical, isotonic composition ranges from 3 to 8 or from 3.5 to 7.5 or from 3.5 to 6.8. In a particular embodiment, the pH of the topical, isotonic composition is from pH of 3.5-5 or from 4-4.5, or from 5-5.5, or from 5-6.8. Topical, isotonic compositions having a pH ranging from pH 3.5 to pH 6.8, are particularly suited for administration to a subject that is aged from puberty to menopause/andropause, e.g., 18 years of age to 50 years of age, 18 years of age to 55 years of age, 18 years of age to 60 years of age, or a child at least one year old. In certain embodiments, the pH of the composition ranges from pH 5 to pH 8. In some embodiments, compositions having a pH ranging from pH 4 to pH 7 may be particularly suited for administration to an infant aged from 0 to 12 months old, a senior adult of at least 60 years of age, an adult (male or female) of reproductive age (e.g., ranging from 18 years to 50 years) who is actively trying to conceive, or both members of a heterosexual dyad which is actively trying to conceive.

The methods provided in the present disclosure may be used on an animal subject (e.g., mammalian, bovine, canine, feline, equine, porcine, ovine, avian, rodent, lagomorph, caprine, non-human primate), preferably a human subject. In certain embodiments, the subject is a male, a female, an intersex subject, a non-binary gendered subject, or a subject of any other gender designation. In certain embodiments, the subject is an infant, a child, or an adult. In certain embodiments, the subject is an adult male of reproductive age (e.g., ranging 18 years to 50 years) that is trying to conceive or adult female of reproductive age (e.g., ranging from 18 years to 50 years) that is trying to conceive. In certain embodiments, the subject is a female in menopause or male in andropause. In certain embodiments, the subject is an infant (aged from 0 to 12 months old), a child at least 1 year old; an adult ranging from 18 years to 50 years of age, 18 to 55 years of age, or 18 to 60 years of age; or a senior adult of at least 50 years, at least 55 years, at least 60 years, or at least 65 years of age. In certain embodiments, a senior is at least 60 years old. In certain embodiments, a senior is at 70 years old. In certain embodiments, a senior is at least 80 years old.

In any of the embodiments provided in the present disclosure, the composition may be administered to an individual subject as part of a treatment regimen, to members of a non-monadic sexual relationship (e.g., sexual dyad, sexual triad) as a part of a treatment regimen, or to both members of a non-sexual dyad. In certain embodiments, the sexual dyad is a homosexual dyad, a heterosexual dyad, or other sexual orientation dyad. In certain embodiments, a non-sexual dyad is parent-child dyad (e.g., mother-child or father-child), caregiver-child dyad, caregiver-adult patient dyad, or caregiver-senior patient dyad.

In some embodiments, the method of optimizing the beneficial microbiome growth in the genital region of a subject in need thereof may comprise application of a pharmaceutical composition to the genital region of the subject or to the genital region of a sexual partner of the subject in need thereof. In some embodiments, optimizing the beneficial microbiome growth comprises inhibiting pathogenic bacterial growth such as growth of Acinetobacter species. In various implementations, the optimizing the beneficial microbiome growth comprises promoting beneficial bacterial growth. Additionally, application of the pharmaceutical composition may have minimal effect to the gametes (e.g., male gametes, female gametes) of the subject or the sexual partner of the subject and/or has minimal effect on zygote formed during intercourse between the subject and a sexual partner. In various implementations, the application may have minimal effect to the male gametes in any genital fluids secreted from the subject or the sexual partner of the subject. For example, the male gametes may have minimal change (e.g. less than 20%) in their motility and/or concentration and/or vitality and/or morphology and/or oxidation-reduction potential and/or sperm DNA fragmentation and/or sperm mitochondrial membrane potential and/or survival and/or sub-cellular alterations (e.g., as compared to gametes produced without application of the composition). These parameters may be measured by any of the methods described herein, and in particular those described in WO 2021/133697 which is hereby incorporated by reference in its entirety.

### Compositions

The present disclosure provides genital products that can optimize genital health and modulate the metabolism of the genital microbiome. In some embodiments, the compositions described herein optimize the penile microbiome. In some embodiments, the compositions described herein optimize the vaginal microbiome of a sexual partner of the user applying the compositions described herein. These topical, isotonic compositions of the present disclosure may include components for supporting growth, dominance, resilience, and metabolism of beneficial lactic-acid producing bacteria and components for inhibiting dysbiotic bacterial growth and metabolism. By influencing the metabolism of the genital microbiome, the growth and metabolic activity of beneficial bacterial species (e.g., Lactobacillus, Streptococcus, Staphylococcus, Corynebacteria) can be enhanced, and the growth and metabolism of bacterial species that lead to genital dysbiosis and disease can be inhibited to restore and maintain genital health. Furthermore, the decrease of certain pathogenic bacteria, such as Acinetobacter species including Acinetobacter baumannii, can be reduced via application of the compositions of the present disclosure. By treating male members of dyads, triads, and higher order social groups of significance, the health of all the members of dyads, triads, or larger social groups can also be improved. Furthermore, people in high stress, crowded, or unhygienic situations, such members of the military, incarcerated individuals, people living in dormitories or shelters, people in man-made or natural disasters can also form a dyad or other social grouping with extensive sharing of anogenital microbiota through daily interactions. People living in such settings can benefit from the improved cleaning and personal care offered by the compositions of the present disclosure.

The pharmaceutical compositions of the present disclosure are typically maintained at an acidic pH. In some embodiments, the composition may be a topical, isotonic compositions comprising a prebiotic oligosaccharide, a metal co-factor, and bornyl acetate (e.g., the composition comprises an essential oil comprising bornyl acetate), at a pH ranging from about 3.5 to about 8. In certain embodiments, the composition may further comprise a pH modifying agent to adjust the final pH of the composition to the target or desired pH. The pH modifying agent may comprise an acidifying agent, an alkalinizing agent, and/or both an acidifying agent and an alkalinizing agent. In certain embodiments, the pH modifying agent is in an amount ranging from 0.01% to 10% (e.g., from 0.01% to 1% or from 0.01% to 0.1% or from 0.1% to 1% or from 1% to 10%) by weight of the composition. Exemplary acidifying agents include, but are not limited to, organic acids such as α-hydroxy acids, citric acid, lactic acid, formic acid, glycolic acid, acetic acid, propionic acid, butyric acid, caproic acid, oxalic acid, maleic acid, benzoic acid, carbonic acid, and the like. Exemplary alkalinizing agents include ammonia, ammonium carbonate, diethanolamine, monoethanolamine, potassium hydroxide, potassium phosphate dibasic, sodium bicarbonate, sodium borate, sodium carbonate, sodium hydroxide, sodium lactate, sodium phosphate dibasic, trolamine, or any combination thereof.

In certain embodiments, the pH of the composition is formulated at a pH to match to a normal, physiological fluid pH (e.g., cervo-vaginal secretions, semen) or the epithelial surface of the genital tissue, or the anogenital epithelium (e.g., mucosa, skin). In certain embodiments, the pH of the composition is less than (e.g., from 3 to) 6.8 or less than 6.5 or less than 6.2 or less than 6.0 or less than 5.7 or less than 5.5, or less than 4.5. In certain embodiments, the pH of the topical isotonic composition is 3.5 to 7.5, 3.5 to 6.8, 3.5 to 6.0, 3.5 to 5.7, 4 to 5, or 4.25 to 4.75. In a particular embodiment, the pH of the topical, isotonic composition is 3.5, 3.8, 4, 4.1, 4.2, 4.25, 4.3, 4.4, 4.5, 4.6, 4.7, 4.75, 4.8, 4.9, 5, 6.5, or 6.8.

In certain embodiments, topical, isotonic compositions of the present disclosure further comprise a buffering agent. The buffering agent may be in an amount ranging from 0.01% to 0.9% by weight of the composition. A buffering agent refers to a compound or a mix of compounds that, when present in a solution, resists changes in the pH of the solution when small quantities of acid or base are added or upon dilution with a solvent or bodily fluid. Buffer capacity is a measure of the resistance to change in the pH of a solution when acids or bases are added to the solution. The total amount of the buffering agent (e.g., conjugate acid-base pair) is selected such that the pH of the composition is maintained at the desired pH or range of pH values. Thus, the greater the amount of the buffering capacity, the more resistant the pH of the composition is to change. In certain embodiments, a buffering agent contains an acidic species to neutralize hydroxide (OH) ions and a basic species to neutralize hydrogen (H) ions. However, the acidic and basic species of the buffering agent should not consume each other through a neutralization reaction.

In certain embodiments, the buffering agent is a simple buffered solution comprising a weak acid and a salt of the weak acid or a weak base and a salt of the weak base. Thus, the buffering agent can include a weak acid-base conjugate pair or weak base-acid conjugate pair. Examples of weak acid/salt of weak acid and weak base/salt of weak base parings include citric acid/sodium citrate, lactic acid/sodium lactate, acetic acid/sodium acetate, monosodium phosphate/disodium phosphate, propionic acid/sodium propionate, butyric acid/sodium butyrate, carbonic acid/sodium bicarbonate, malic acid/sodium malate, ascorbic acid/sodium ascorbate benzoic acid/sodium benzoate, succinic acid/sodium succinate and sodium borate/boric acid. In certain embodiments, the buffering agent comprises unrelated weak acid-base pairs. Examples of such combinations include disodium phosphate/citric acid, disodium phosphate/lactic acid, monosodium phosphate/sodium lactate, monosodium phosphate/sodium citrate, sodium citrate/lactic acid, sodium lactate/citric acid, monopotassium phosphate/citric acid, monopotassium phosphate/lactic acid, monopotassium phosphate/sodium lactate, monopotassium phosphate/sodium citrate, monopotassium citrate/lactic acid, and potassium lactate/citric acid. In addition, for multivalent anions, the calcium salt rather than sodium salt may be used (e.g., calcium citrate). Example buffer can also include gluconolactone/gluconic acid.

In certain embodiments, the buffering agent is selected such that the buffering agent's acid form has a pKa the same as or close to the desired pH of the composition or a pH within the desired range of pH values, functions with a similar buffering capacity to surfaces and fluids that physiologically occur in genital region (e.g., vaginal mucin-acidic barrier, cervico-vaginal secretions, semen, menses-flow, or a combination thereof), or maintains pH to that of the target epithelial surface (Rastogi et al., Contraception. 93:337, 2016). In certain embodiments, a buffering agent comprises a monocarboxylate, a dicarboxylate, a carboxylic acid, or a combination thereof. In some embodiments, a buffering agent may comprise an acetate, borate, citrate, fumarate, lactate, malate, malonate, nitrate, phosphate, propanoate, succinate, tartrate, tromethamine, or any combination thereof. In some embodiments, a buffering agent comprises lactic acid, sodium lactate, sodium phosphate (monobasic, dibasic, or both), potassium phosphate (monobasic, dibasic, or both), sodium citrate, potassium citrate, calcium citrate, acetic acid, sodium acetate, citric acid, disodium citrate, trisodium citrate, boric acid/sodium, succinic acid, sodium succinate, gluconolactone, disodium succinate, tartaric acid, sodium tartrate, sodium ascorbate, ascorbic acid, tromethamine (Tris), or any combination thereof. In certain embodiments, the buffering agent in the compositions may comprise citric acid, a sodium phosphate such as monosodium phosphate and/or disodium phosphate, lactic acid and sodium lactate, gluconolactone, or mono- or disodium phosphate and lactic acid.

In certain embodiments, the compositions of the present disclosure may further comprise a preservative. The preservative may be in an amount of 0.001% to 4% (e.g., 0.001% to 1%) by weight of the composition. Exemplary preservatives include, but are not limited to, caprylyl glycol, cranberry extract, dichlorobenzyl alcohol, gluconolactone, green tea extract, oleuropein, pentylene glycol, phenethyl alcohol, pomegranate extract, potassium benzoate, propanediol, resveratrol, hydantoin, benzoic acid, benzyl alcohol, dehydroacetic acid, ethylhexyl glycerin, Lactobacillus ferment, pentylene glycol, potassium sorbate, sodium benzoate, sodium dehydroacetate, glyceryl caprylate, sodium salicylate, Euxyl® K 903 (Benzoic Acid 11-13%; Dehydroacetic acid 6.5-7.5%; Benzyl alcohol 78-84%, Lincoserve™ BDHA (Dehydroacetic acid 7.5-8.5%; Benzyl alcohol 86-88%, Lincoserve™ WpH-LO (Pentylene Glycol 30-40%; Propanediol 25-35%; Ethylhexyl Glycerin 1-10%; Caprylyl Glycol 10-20%, Linatural™ Ultra-3 (Pentylene Glycol 30-45%; Propanediol 45-55%; Phenethyl Alcohol 5-15%, Linatural™ MBS-1 (Ethylhexyl Glycerin 1-10%; Propanediol 75-85%; Potassium Sorbate 1-10%, Lincoserve™ SSB (water 55-65%; Sodium Benzoate 20-30%; Sodium Salicylate 10-20, or any combination thereof. In various implementations, the preservative may be one or more α-hydroxy acids such as lactic acid, citric acid, glycolic acid, or combinations thereof.

In certain embodiments, the topical, isotonic compositions of the present disclosure are sterile and/or preservative-free (e.g., less than 0.1%, or less than 0.01%, or less than 0.001% by weight of the composition). Although lower pH may work synergistically to improve the efficacy of preservatives (e.g., in terms of minimizing or inhibiting microbial growth), acidic compositions having preservatives may not have a similar effect on gametes (see Examples). Some acidic compositions (e.g., pH 4-5) having preservatives have been shown to have less effect on gametes such as sperm than those at higher pH (e.g., pH 6.5-7.5). In some embodiments, the preservative may be added in an amount to provide anti-microbial efficacy of pathogenic growth including bacterial or fungal growth. In some embodiments, the preservative decreases the growth of pathogenic organisms such as species from the genus Acinetobacter including Acinetobacter baumannii or other pathogenic organisms such as E. coli, P. aeruginosa, S. aureus, A. brasiliensis, C. albicans or combinations thereof in the composition.

Pharmaceutical compositions such as topical, isotonic compositions of the present disclosure may comprise a prebiotic oligosaccharide in an amount ranging from 0.001% to 5% by weight of the composition. Prebiotic oligosaccharide refers to oligosaccharides substrates that induce the growth or activity of beneficial microorganisms of the microbiota, e.g., genital microbiota. In certain embodiments, a prebiotic is non-digestible and resistant to breakdown by stomach acid and enzymes in the human gastrointestinal tract, selectively fermented by genital microbiota (e.g., beneficial genital microbiota), selectively target and stimulate the growth and activity of specific genital microbiota (e.g., healthy and/or beneficial genital microbiota), or any combination thereof.

One or more prebiotics may be added to composition. Suitable prebiotics may include oligosaccharides, particularly galactooligosaccharide (GOS), palatinoseoligosaccharide, soybean oligosaccharide, gentiooligosaccharide, xylooligomers, non-degradable starch, lactosaccharose, lactulose, lactitol, maltitol, polydextrose, or the like. Examples of prebiotic oligosaccharides that may be used in the compositions of the present disclosure include raffinose, lactulose, trehalose, galactooligosaccharide, alpha-glucan oligosaccharide, beta-glucan oligosaccharide, maltose, xylose, fructooligosaccharide, isomaltooligosaccharide, inulin, pectin, or any combination thereof. In certain embodiments, a prebiotic oligosaccharide does not include xylose.

The compositions of the present disclosure may also comprise a metal co-factor. Metal co-factors may be metallic ions, or salts thereof, which often act as a catalyst for an enzyme's activity. Specifically, the metal co-factors may assist enzymes involved in glycosylation of proteoglycans, such as glycosaminoglycans, which are involved in a variety of physiological functions including barrier immune protection, epithelial hydration and providing viscosity to natural bodily fluids, such as cervico-vaginal secretions, penile foreskin secretions, smegma, distal urethral sections, and semen. The metal co-factor may be requisite in some conditions for Lactobacillus growth. Pathogenic bacteria may sequester such metal co-factors, as an act of establishing dominance over beneficial genital microbiota. The metal co-factor may be present in an amount ranging from 0.0001% to 0.1% by weight of the composition. A metal co-factor may comprise zinc, selenium, molybdenum, manganese, cobalt, iron, copper, including salts thereof, or any combination thereof. The metal co-factor may be added to the composition as a salt of the co-factor metallic ion comprising a counterion, for example, the chloride salt. For example, the metal co-factor may comprise Mn²⁺, Mn³⁺, Mn⁴⁺, Mn⁶⁺, Mn⁷⁺, Zn¹⁺, Zn²⁺, Se²⁺, Se⁴⁺, Se⁶⁺, Co²⁺, Co³⁺, Fe²⁺, Fe³⁺, Cu¹⁺, Cu²⁺, or pharmaceutically acceptable salts (e.g. the chloride salt) thereof, or hydrates of any of the foregoing. In some embodiments, the metal co-factor is manganese (II) chloride. It will be understood that some hydrates of these metallic salts may dissociate to form the metal-cofactor in the topical isotonic solutions.

The compositions may include borneol, pharmaceutically acceptable salts, esters, and prodrugs thereof. For example, the compositions may comprise bornyl acetate. Bornyl acetate is a compound commonly found in pine needles, valerian root, fir needles, hemlock, cypress, rosemary, and occasionally juniper berries and spearmint. Bornyl acetate has acetylcholinesterase enzyme inhibitory, anti-inflammatory, and analgesic activity as shown for several indications in described in L Yang, et al., Biomed Pharmacother 103 (2018): 234-239, S. Lu et al. Biomed Res Int 2018 (2018): 3589874, T Zhang et al. Front Pharmacol 8 (2017): 786, and D Szwajgier et al., Curr Alzheimer Res 16 (2019): 963, each hereby incorporated by reference in their entirety. In some embodiments, the pharmaceutical composition comprises bornyl acetate or pharmaceutically acceptable salts or prodrugs thereof. In some embodiments, the pharmaceutical composition comprises borneol or pharmaceutically acceptable salts thereof or prodrugs (e.g., ester prodrugs) of any of the foregoing.

In some embodiments, the compositions of the present disclosure may comprise more than 0.0001% bornyl acetate by weight of the composition (e.g., more than 0.00025% bornyl acetate, from 0.0001% to 1% bornyl acetate, from 0.0001% to 0.5% bornyl acetate, from 0.0001% to 0.3% bornyl acetate, from 0.0001% to 0.2% bornyl acetate, from 0.0001% to 0.1% bornyl acetate, from 0.0001% to 0.001% bornyl acetate, from 0.001% to 0.01% bornyl acetate, from 0.01% to 0.1% bornyl acetate, from 0.1% to 1% bornyl acetate, from 0.01% to 0.3% bornyl acetate, from 0.1% to 0.3% bornyl acetate, from 0.00025% to 0.20% bornyl acetate, 0.0005% to 0.20% bornyl acetate, from 0.0001% to 0.15% bornyl acetate, from 0.00025% to 0.15% bornyl acetate, from 0.001% to 0.06% bornyl acetate) by weight of the composition. In some embodiments, the composition may comprise less than 0.3% (e.g., less than 0.15%, less than 0.015%, from 0.0001% to 0.015%, from 0.00025% to 0.015%) bornyl acetate by weight of the composition. In some embodiments, the compositions of the present disclosure may comprise more than 0.0001% borneol, pharmaceutically acceptable salts thereof, esters (e.g., C₁-C₇ esters), or prodrugs of any of the foregoing (e.g., bornyl acetate) by weight of the composition (e.g., more than 0.00025% borneol, pharmaceutically acceptable salts thereof, or prodrugs of any of the foregoing, from 0.0001% to 1% borneol, pharmaceutically acceptable salts thereof, or prodrugs of any of the foregoing, from 0.0001% to 0.5% borneol, pharmaceutically acceptable salts thereof, or prodrugs of any of the foregoing, from 0.0001% to 0.3% borneol, pharmaceutically acceptable salts thereof, or prodrugs of any of the foregoing, from 0.0001% to 0.2% borneol, pharmaceutically acceptable salts thereof, or prodrugs of any of the foregoing, from 0.0001% to 0.1% borneol, pharmaceutically acceptable salts thereof, or prodrugs of any of the foregoing, from 0.0001% to 0.001% borneol, pharmaceutically acceptable salts thereof, or prodrugs of any of the foregoing, from 0.001% to 0.01% borneol, pharmaceutically acceptable salts thereof, or prodrugs of any of the foregoing, from 0.01% to 0.1% borneol, pharmaceutically acceptable salts thereof, or prodrugs of any of the foregoing, from 0.1% to 1% borneol, pharmaceutically acceptable salts thereof, or prodrugs of any of the foregoing, from 0.01% to 0.3% borneol, pharmaceutically acceptable salts thereof, or prodrugs of any of the foregoing, from 0.1% to 0.3% borneol, pharmaceutically acceptable salts thereof, or prodrugs of any of the foregoing, from 0.00025% to 0.20% borneol, pharmaceutically acceptable salts thereof, or prodrugs of any of the foregoing, 0.0005% to 0.20% borneol, pharmaceutically acceptable salts thereof, or prodrugs of any of the foregoing, from 0.0001% to 0.15% borneol, pharmaceutically acceptable salts thereof, or prodrugs of any of the foregoing, from 0.00025% to 0.15% borneol, pharmaceutically acceptable salts thereof, or prodrugs of any of the foregoing, from 0.001% to 0.06% borneol, pharmaceutically acceptable salts thereof, or prodrugs of any of the foregoing) by weight of the composition. In some embodiments, the composition may comprise less than 0.3% borneol, pharmaceutically acceptable salts thereof, or prodrugs of any of the foregoing (e.g., less than 0.15% borneol, pharmaceutically acceptable salts thereof, or prodrugs of any of the foregoing, less than 0.015% borneol, pharmaceutically acceptable salts thereof, or prodrugs of any of the foregoing, from 0.0001% borneol, pharmaceutically acceptable salts thereof, or prodrugs of any of the foregoing to 0.015% borneol, pharmaceutically acceptable salts thereof, or prodrugs of any of the foregoing, from 0.00025% borneol, pharmaceutically acceptable salts thereof, or prodrugs of any of the foregoing to 0.015% borneol, pharmaceutically acceptable salts thereof, or prodrugs of any of the foregoing), by weight of the composition.

Borneol, bornyl acetate, pharmaceutically acceptable salts thereof, or prodrugs of any of the foregoing typically have at least one chiral center. The compositions may comprise the enantiomerically pure compound, enantiomeric mixtures of an indicated compound, or racemic mixtures of the enantiomer. For example, bornyl acetate may be present as (+)-bornyl acetate, (−)-bornyl acetate, racemic mixtures thereof, or enantiomeric mixtures thereof (e.g., a weight ratio of (+)-bornyl acetate to (−)-bornyl acetate of from 100:1 to 1:100 or 50:1 to 1:50 or 25:1 to 1:25 or 10:1 to 1:10).

In some embodiments, the compositions of the present disclosure may also comprise an essential oil which comprises bornyl acetate, a monoterpene ester, in an amount ranging from 0.005% to 0.5% by weight of the composition. In certain embodiments, the essential oil comprising bornyl acetate may be an essential oil selected from Juniperus communis, Juniperus occidentalis, Juniperus scopulorum, Abies sibirica, Abies alba, Abies balsamea, Abies fraseri, Abies grandis, Abies spectabilis, Abies koreana, Abies procera, Abies nordmanniana, Abies magnifica, Abies pinsapo, Abies lasiocarpa, Abies concolor, Pseudotsuga menziesii, Ambrosia trifida, Pinus mugo, Romanian solidago, Ribes nigrum, Laurus nobilis, Rosmarinus officinalis, or any combination thereof. In some embodiments, the essential oil comprising bornyl acetate comprises bornyl acetate in an amount of at least 5% by weight of the essential oil. In some embodiments, the essential oil comprising bornyl acetate comprises bornyl acetate in an amount ranging from 5% to 30% or from 10% to 30% by weight of the essential oil. Accordingly, in some embodiments, the topical, isotonic compositions may comprise more than 0.00025% bornyl acetate (e.g., from 0.00025% to 0.15% bornyl acetate, 0.0005% to 0.15% bornyl acetate) by weight of the composition. In some embodiments, the topical isotonic compositions may comprise from 0.0001% bornyl acetate to 0.3% bornyl acetate by weight of the composition. In certain implementations, the topical isotonic compositions may comprise an essential oil comprising bornyl acetate such that from 0.0001% bornyl acetate to 0.3% bornyl acetate (derived from the essential oil) by weight of the composition.

In certain embodiments, compositions of the present disclosure further comprise a bisabolene. The bisabolene may be in an amount ranging from 0.0001% to 0.1%. Bisabolene has anti-inflammatory, wound healing, skin strengthening, anti-tumor and/or analgesic activity. In certain embodiments, bisabolene is obtained from Commiphora guidotti, Pallines spinosa, Platanus chiapensis, Platanus gentryi, Platanus kerrii, Platanus mexicana, Platanus oaxacana, Platanus occidentalis, Platanus orientalis, Platanus racemosa, Platanus rzedowskii, Platanus wrightii, Platanus acerifolia, or any combination thereof.

The compositions of the present disclosure may further comprise a flavonoid. The flavonoid may be in an amount ranging from 0.001% to 0.1%. Flavonoids possess a wide range of biological and pharmaceutical activities, including antioxidant and anti-inflammatory activities. In certain embodiments, the flavonoid comprises catechin, epicatechin, rutin, luteolin, apigenin, kaempherol, myricetin, quercetin, quercitrin, naringin, naringenin, hesperetin, hesperidin, taxifolin, genistin, genistein, daidzein, cyanidin, apigenidin, tangeritin, fisetin, galangin, isorhamnetin, pachypodol, rhamnazin, pyranoflavonol, fluranoflavonol, eriodictyol, homoeriodictyol, taxifolin, gallocatechin, catechin 3-gallate, gallocatechin 3-gallate, epigallocatechin, epicatechin 3-gallate, epigallocatechin 3-gallate, theaflavin, thearubigin, proanthocyanidin, dephinidin, malvidin, pelargonidin, peonidin, petunidin, isoflavone, glycitein, isoflavane, isoflavandiol, isoflavene, coumestan, pterocarpan, myricitrin, phloridzin, or any combination thereof. In certain embodiments, the flavonoid comprises a citrus essential oil (e.g., Citrus reticulata) as disclosed in Y Yang, et al., J Food Sc 12 (217): 2840-2846, hereby incorporated by reference in its entirety, wherein monoterpene hydrocarbons comprise at least 75% or at least 85% of the total composition of the essential oil. Monoterpene concentration may be measured using the method as described in Njoroge et al. (Journal of Essential Oil Research 18:659, 2006) hereby incorporated by reference in its entirety.

In certain embodiments, compositions of the present disclosure may further comprise a sesquiterpene alcohol and/or monocyclic sesquiterpene. The sesquiterpenes may be in an amount ranging from 0.001% to 0.1% by weight of the composition. Sesquiterpenes possess a wide range of biological and pharmaceutical activities, including increasing skin hydration, antimicrobial activity, and antifungal activity. In certain embodiments, the sesquiterpenes may include nerolidol, an essential oil from Citrus aurantum var sp, bisabolol, patchoulol, alpha-santalol, zingiberene, or combinations thereof. In some embodiments, the sesquiterpene alcohol or monocyclic comprises nerolidol, an essential oil from Citrus aurantum (e.g., Citrus aurantium var. amata) and/or Citrus bigaradia, bisabolol, patchoulol, alpha-santalol, zingiberene, or combinations thereof. In certain implementations the sesquiterpene alcohol and/or monocyclic sesquiterpene is neroli oil.

The composition may further comprise a biofilm inhibiting agent. The biofilm inhibiting agent may be in an amount ranging from 0.001% to 0.16%. In certain embodiments, the biofilm inhibiting agent comprises Lamiaceae essential oil, Garcinia extract, Eurycoma longifolia extract, or any combination thereof. Examples of Lamiaceae essential oil include essential oil from Mentha spicata, Mentha pulegium, Mentha piperita, Mentha aquatica, Mentha arvensis, Mentha asiatica, Mentha australis, Mentha canadensis, Mentha cervina, Mentha citrata, Mentha crispata, Mentha dahurica, Mentha diemenica, Mentha laxiflora, Mentha, longifolia, Mentha requienii, Mentha sachalinensis, Mentha satureioides, Mentha suavenolens, Mentha vagans, Melissa officinalis, Monarda Fistulosa, or any combination thereof. Garcinia plants are sources of antioxidant xanthones, which possess anti-microbial and anti-inflammatory properties. Examples of xanthones include y-mangostin, garcinone-D, gartanin, and smeathxanthone. Examples of Garcinia extract include extract from Garcinia mangostana, Garcinia travancorica, Garcinia cambogia, Garcinia kola, Garcinia zeylanica, Garcinia xanthochymus, or any combination thereof.

In certain implementations, compositions of the present disclosure further comprise a cell membrane active phytosterol. The phytosterol may be in an amount ranging from 0.001% to 0.1%. Phytosterols may inhibit the growth of pathogenic bacteria and have the potential to inhibit the activity of pore-forming toxins such as vaginolysin, leukotoxin, and other cholesterol dependent cytotoxins found in pathogenic strains of bacteria such as some Staphylococcus, Clostridium, and Gardnerella spp. In certain embodiments, the phytosterols comprise apigenin, β-sitosterol, campesterol, brassicasterol, stigmasterol, sitosterol, or any combination thereof. Examples of phytosterol sources include ginseng (Panax quinquefolium) seed extract, carrot, yam or coriander extract, ginger root extract, Mirabilis Jalap, or any combination thereof.

In certain embodiments, compositions of the present disclosure further comprise a prebiotic spice extract. The prebiotic spice extract may be in an amount ranging from 0.001% to 0.02% by weight of the composition. Exemplary prebiotic spice extracts include extract from ginger, black pepper, cayenne pepper, cinnamon, oregano, rosemary, turmeric, or any combination thereof.

The compositions of the present disclosure may also comprise a viscosity-increasing agent. Viscosity is a property of liquids that is closely related to the resistance to flow. It may be defined by Couette flow, which is the laminar flow of a viscous fluid in the space between two parallel plates, one of which is moving relative to the other. The flow is driven by virtue of viscous drag force acting on the fluid and the applied pressure gradient parallel to the plates.

The compositions may comprise one or more rheology agents. In some embodiments, the composition may comprise one or more non-cellulose based rheology agents. In some embodiments, the topical, isotonic composition comprises a rheology agent selected from poloxamers, polyacrylics (e.g. polyacrylic acids), polybutene, polycarbophil, polyvinyl alcohol, polyvinylpyrrolidone polymers, polyoxazoline polymers, and combinations thereof.

In some embodiments, the compositions of the present disclosure further comprise one or more humectants such as glycerin, hexylene glycol, arabinogalactan, caprylyl glycol, or combinations thereof. In certain embodiments, the compositions may comprise a humectant that is an extract of a plant from the genus Monotropa (e.g., Monotropa hypopitys) such as those described in JP App No 2009191075 A to Yamada, hereby incorporated by reference in its entirety.

In some embodiments, the compositions of the present disclosure further comprise one or more anti-inflammatory agents and/or one or more soothing agents. Specific anti-inflammatory and/or soothing agents include ICAM inhibitors (e.g., CD11a, ezrin (EZR), CD18, glycyrrhetinic acid, pyrrolidinedithiocarbamate), NFκB inhibitors (e.g., (heterocyclic thiazole, lipoic acid, efalizumab, 4-[(4-Methylphenyl)thio]thieno[2,3-c]pyridine-2-carboxamide, silibinin, stilbenes, (+)-epigalloylcatechin-gallate [(+)-EGCG]), cytokine inhibitors TSLP inhibitors, IL-25 inhibitors, IL-33 inhibitors, IL-1 inhibitors, TNF inhibitors (e.g., TNF-α inhibitors, TNF-β inhibitors), quercetin and isoforms thereof (e.g., isoquercetin, etc.), non-steroidal anti-inflammatory drugs (e.g., aspirin), and extracts from plants of the genus Vigna (e.g., Vigna caracalla), extracts of plants from the genus Rhododendron (e.g., Rhododendron aceae), extracts of plants from the subfamily Monotropaceae such as Allotropa virgate as disclosed in JP 2009191075 to T Yamada, hereby incorporated by reference in its entirety, and combinations thereof. In various embodiments, the anti-inflammatory agent and/or soothing may be present in an amount of from 0.0001 to 10% (e.g. from 0.001 to 5%) by weight of the composition

The extracts may be prepared by enzymatic extraction, solvent extraction, steam distillation, or any other method known in the art. In some embodiments, at least one of the topical compositions of the invention comprises an extract, obtained by steam distillation, of any of the forgoing plants and biological materials (each one being considered a distinct embodiment). In some embodiments, at least one of the topical compositions of the invention comprises an extract, obtained by extraction with water (e.g., basic, neutral, or acid), of any of the forgoing plants and biological materials (each one being considered a distinct embodiment). The water of extraction may further include a co-solvent miscible with water, including lower alcohols (e.g., C₁₋₆), such as methanol, ethanol, isopropanol, propanol, butanol (typically, ethanol). In some embodiments, at least one of the topical compositions of the invention comprises an extract, obtained by extraction with a solvent system comprising from 5-95% (v/v) or 10-90% (v/v) or 20-80% (v/v) or 40-60% (v/v) water (e.g., basic, neutral, or acid) and 5-95% (v/v) or 10-90% (v/v) or 20-80% (v/v) or 40-60% (v/v) ethanol, of any of the forgoing plants and biological materials (each one being considered a distinct embodiment). In some embodiments, at least one of the topical compositions of the invention comprises an extract, obtained by extraction with an organic solvent (e.g., non-polar, polar aprotic, or polar protic), of any of the forgoing plants and biological materials (each one being considered a distinct embodiment). Suitable solvents include hexane and other C₁₋₁₂ or C₅₋₈ hydrocarbons, lower alcohols, C₂₋₁₆ ethers (e.g., diethyl ether), C₃₋₁₂ esters (e.g., ethyl acetate), C₂₋₁₂ (e.g., acetone, butanone), carbon dioxide (liquid or supercritical) The biological extracts may be dried under vacuum or atmospheric pressure to remove water and solvents of extraction. The biological extracts may be dried by lyophilization. The biological extracts may be passed over activated carbon or charcoal and/or passed through filters and/or microfilters to remove bacteria and other biological materials.

In certain embodiments, compositions of the instant disclosure are formulated to have viscosity best suited for the target tissue (e.g., anogenital region) and to mimic the properties of normal genital fluids. For example, compositions formulated as gels applied to mucous membranes may be designed to have viscosity values consistent with or similar to normal mucus, and exhibiting non-Newtonian, shear-thinning (pseudoplastic) flow properties. Standardized methodology for quantitative comparisons of over-the-counter vaginal products' base features such as, stickiness, ropiness, peaking, rubberiness, thickness, smoothness, and slipperiness, are known in the art (Mahan et al., Contraception, 84:184, 2011). In some embodiments, compositions formulated as gels applied to mucous membranes may strengthen mucus quality and/or mucin coverage of the body surface. In certain implementations, the compositions may stimulate the production or proffer an acidic barrier in the urogenital and/or anogenital region with improved muco-adhesion, which may increase the bioavailability of one or more active components of the composition and result in a beneficial impact on the genital microbiome as disclosed in N Peppas, et al., J Biomater Sci Polym Ed. 20 (2209): 1-20, hereby incorporated by reference in its entirety and particularly in relation to muco-adhesive carrier development. In certain embodiments, the compositions of the present disclosure may include a specific buffer which allows for increased buffering capacity in the vagina at a pH range of 5-7.

For compositions applied to skin (such as the penis, vulva, perineum) or inside the vagina, a viscosity-increasing agent can be added in an amount that allows the composition to spread easily to form a thin layer when minimal physical pressure is applied, and to have adequate viscosity and shear-thinning properties so that the composition does not “run” off or out of the genital tissue upon topical application. Mucoadhesive formulations that are retained at the genital surface (e.g., vulvar, vaginal, penile, foreskin surface) for prolonged biological activity are known in the art (reviewed by Khutoryanskiy, Macromol. Biosci. 11:748, 2011; Brooks, Front. Chem. 3:65, 2015). Muco-adhesive formulas must have polymer compositions that actively admix and interact with physiologic mucin and mucus of secretions. Some common gelling agents do not intertwine with natural mucins and are therefore not muco-adhesive and are rapidly lost from the epithelium. In certain embodiments, compositions of the instant disclosure are formulated to have viscosity best suited for the target tissue (e.g., penile region) and to mimic the properties of normal genital fluids. Standardized methodology for quantitative comparisons of over-the-counter products may be based features such as, stickiness, ropiness, peaking, rubberiness, thickness, smoothness, and slipperiness, are known in the art.

Compositions of the present disclosure may comprise a viscosity-increasing agent in an amount ranging from, for example, 0.05% to 10% by weight of the composition. In certain embodiments, the viscosity enhancing agent comprises a tensioactive cellulose or gum. Tensioactive celluloses and gums can also act to emulsify and pull particles and essential oils into solution. In certain embodiments, additional surfactants, which may have a harsh effect on cells, are not needed or included in the compositions of the present disclosure. For example, the composition may be substantially free of surfactants such as having less than 1% or less than 0.1% or less than 0.01% surfactants by weight of the composition. In certain embodiments, the viscosity-increasing agent comprises guar gum, methylcellulose, ethylcellulose, ethyl methyl cellulose, ethyl hydroxyethyl cellulose, carboxymethylcellulose, hydroxypropylcellulose, hydroxyethyl methyl cellulose, hydroxypropylmethylcellulose (hypromellose), hydroxyethylcellulose, cetyl hydroxyethycellulose, hydroxypropyl guar gum glycosaminoglycans (e.g., hyaluronic acid), pullulan, nonionic triblock copolymers such as poloxamers, gelatins, alginates, carrageenan, and agar, or any combination thereof. In some embodiments, the compositions may comprise a viscosity-increasing agent comprising glycosaminoglycans (e.g., hyaluronic acid), polyacrylic acids, nonionic triblock copolymers such as poloxamers, gelatins, carrageenan, agar, and combinations thereof.

Compositions having a pH of 3.5 to 6, may be particularly suited for administration to a subject that is aged from adolescence to menopause/andropause, e.g., 18 years of age to 50 years of age, or a child at least one year old. In certain embodiments, an isotonic, topical composition for administration to a subject of more than 12 years of age (e.g., from 12 years of age to 50 years of age, more than 50 years of age, more than 60 years of age, more than 70 years of age, more than 80 years age, more than 90 years of age, more than 100 years of age) has a pH in the urogenital and/or anogenital region of from 3.8 to 4.8. In certain embodiments, an isotonic, topical composition for administration to a child at least one year old has a pH in the anogenital and/or urogenital region of 5. Topical, isotonic compositions having a pH of 4.8 to 7 are particularly suited for administration to an infant aged less than 12 months old, to a subject of 18 years of age to 50 years of age who is trying to conceive a child, or a senior of at least 60 years of age. In certain embodiments, a topical, isotonic composition for administration to an infant has a pH in the anogenital and/or urogenital region of 6.5. In certain embodiments, a topical, isotonic composition for administration to a subject of 18 years of age to 50 years of age who is trying to conceive a child has a pH in the anogenital and/or urogenital region of from 6.5 to 7.

Topical compositions of the present disclosure may be isotonic to the target genital fluids or tissues that they will contact. Tonicity is a measure of the effective osmotic pressure gradient (as defined by the water potential of two solutions) of two solutions separated by a semipermeable membrane. Tonicity is commonly used when describing the response of cells immersed in an external solution. In other words, tonicity is the relative concentration of solutions that determine the direction and extent of diffusion from a fluid across cell membranes in tissue. Blood normally has an osmotic pressure that corresponds to that of a 0.9% solution of sodium chloride. A composition (e.g., solution or gel) is considered isotonic when its tonicity matches that of the physiologic fluids it will contact. A composition is isotonic with a body fluid when the magnitude of the salts (ions) are equal between the composition and the physiologic fluid. Tonicity equilibrium is reached in physiologic fluids by water moving across cell membranes, but the salts and ions staying in their fluid of origin. A solution is isotonic with a living cell if there is no net gain or loss of water by the cell, or other changes in the cell ultrastructure, when it is in contact with said solution, even though individual water molecules may move freely across the cell membranes.

Hypertonic solutions cause a net movement of water out of the cells (as the water moves to create equilibrium with the high salt levels outside of the cell). This dehydration of the cell is concentration dependent and leads to osmotic stress which can increase reactive oxygen species, cause cytoskeletal rearrangement, and damage DNA and mitochondrial function within minutes of exposure. Most current genital products are hypertonic, resulting in epithelial cell and sperm death on contact. Hypotonic solutions cause a net flow of water into the cell and cause cell bursting and death. Some deviations of salt levels in physiologic fluids from the level found in blood and tissues may serve a purpose. For example, the lower osmolality of cervico-vaginal fluids that facilitates vaginal epithelial cell lysis and death as a part of normal vaginal function. In another example, the higher osmolality of semen can protect sperm cells from the lower osmolality of cervico-vaginal secretions following ejaculation in the vagina and admixing of fluids during vaginal intercourse.

Related to tonicity is osmosis, which is the movement of solvent across a semipermeable membrane from an area of higher solute concentration to an area of lower solute concentration to produce equilibrium. Osmotic pressure of a solution is the pressure that must be applied to stop the flow of solvent across a semipermeable membrane. In certain embodiments, the compositions of the present disclosure further comprise an osmolality adjusting agent to adjust the tonicity of the compositions. Exemplary osmolality adjusting agents include electrolytes, mono- or disaccharides, inorganic salts (e.g., sodium chloride, calcium chloride, potassium chloride, sodium sulfate, magnesium chloride), or a combination thereof. In some embodiments, an osmolality adjuster is glucose, sucrose, sodium chloride, potassium chloride, calcium chloride, sodium sulfate, magnesium chloride, dextrose, mannitol, or any combination thereof.

In certain embodiments, the osmolality range of the compositions disclosed herein ranges from 120 mOsm/kg to 450 mOsm/kg or from 240 mOsm/kg to 450 mOsm/kg. In certain embodiments, the osmolality of the compositions of the present disclosure is 120 mOsm/kg, 125 mOsm/kg, 130 mOsm/kg, 135 mOsm/kg, 140 mOsm/kg, 145 mOsm/kg, 150 mOsm/kg, 155 mOsm/kg, 160 mOsm/kg, 165 mOsm/kg, 170 mOsm/kg, 175 mOsm/kg, 180 mOsm/kg, 185 mOsm/kg, 190 mOsm/kg, 195 mOsm/kg, 200 mOsm/kg, 205 mOsm/kg, 210 mOsm/kg, 215 mOsm/kg, 220 mOsm/kg, 225 mOsm/kg, 230 mOsm/kg, 235 mOsm/kg, 240 mOsm/kg, 245 mOsm/kg, 250 mOsm/kg, 255 mOsm/kg, 260 mOsm/kg, 265 mOsm/kg, 270 mOsm/kg, 280 mOsm/kg, 285 mOsm/kg, 290 mOsm/kg, 295 mOsm/kg, 300 mOsm/kg, 305 mOsm/kg, 310 mOsm/kg, 315 mOsm/kg, 320 mOsm/kg, 325 mOsm/kg, 330 mOsm/kg, 335 mOsm/kg, 340 mOsm/kg, 345 mOsm/kg, 350 mOsm/kg, 355 mOsm/kg, 360 mOsm/kg, 365 mOsm/kg, 370 mOsm/kg, 375 mOsm/kg, 380 mOsm/kg, 385 mOsm/kg, 390 mOsm/kg, 395 mOsm/kg, 400 mOsm/kg, or 450 mOsm/kg.

In certain embodiments, the topical, isotonic composition is matched for tonicity (e.g., salt/ion levels) to the normal, physiological genital fluid pH (e.g., CVF, urethral secretions, semen, smegma) of the subject; genital tissue of the subject (e.g., vaginal mucosa, genital skin); or at an appropriate tonicity for the particular method of use (e.g., for use during fertile window in a subject or sexual dyad that is trying to conceive). In certain embodiments, the tonicity ranges from 125 mOsm/kg to 240 mOsmo/kg. Such embodiments match the hypotonic CVF which supports lysis of vaginal epithelial cells and vaginal “self-cleaning.” This cell lysis releases glycogen, which healthy genital microbiota utilize for growth and development. Such embodiments are ideal for delivery inside the vaginal canal. In other embodiments the tonicity ranges from 240 mOsm/kg to 280 mOsm/kg. Such embodiments match the tonicity of genital tissues and are ideal for contact with skin genital tissue surfaces. In a particular embodiment, the tonicity ranges from 280 mOsmo/kg to 450 mOsmo/kg to match the tonicity of semen as deposited in the vagina. In certain embodiments, tonicity may be expressed as mOsm/kg or mOsm/L. In certain embodiments, the osmolality and osmolarity values are substantially interchangeable.

In certain embodiments, the topical, isotonic composition further comprises a solvent (e.g., aqueous solvent, water) in an amount greater than 88% (e.g., ranging from 88% to 98%). In further embodiments, the solvent comprises water.

In certain embodiments, the topical, isotonic composition further comprises an additional therapeutic agent. The additional therapeutic agent may improve cell or tissue function or treat an underlying disease or disorder. In certain embodiments, the therapeutic agent is an antibiotic, anti-fungal agent, anti-viral agent, or any combination thereof. Exemplary anti-fungal agents include butoconazole nitrate, clotrimazole, miconazole nitrate, terconazole, tioconazole, econazole nitrate, efinaconazole, ketoconazole, luliconazole, naftifine hydrochloride, oxiconazole nitrate, sertaconazole nitrate, sulconazole nitrate, tavaborole, terbinafine, acyclovir, tenovir, zidovudine, stavudine, metronidazole, or a combination thereof. In some embodiments, the additional therapeutic agent is a vaccine (e.g., multivalent vaccine) to provide immunity against a viral or bacterial disease. Suitable vaccines include uropathogenic Escheri coli bacteria as disclosed in W Hopkins, et al., J Urol., 177 (2007): 1349-1353, hereby incorporated by reference in its entirety, and particularly in relation to the vaccine suppositories used in the study. In some embodiments, the vaccine may include a cholera vaccine such as those as disclosed in P Kozlowski, et al., Infection and Immunity 65 (1997): 1387-1394, which is hereby incorporated by reference in its entirety and particularly in relation to cholera vaccines, and vaccines containing killed Vibrio cholerae cells. In some embodiments, the vaccine may include a SARS-CoV-2 or COVID-19 vaccine. In some embodiments, the additional therapeutic agent may treat or prevent atrophic vaginitis. Suitable agents for the treatment or prophylaxis of atrophic vaginitis include hyaluronic acid, estrogens including estradiol-17β, conjugated estrogens, estradiol hemihydrate, dehydroepiandrosterone, estradiol acetate, selective estrogen receptor modulators, including bazedoxifene, cyclofenil, lasofoxifene, ormeloxifene, ospemifene, raloxifene, toremifene, and combinations thereof.

In certain embodiments, the additional therapeutic agent is a topical pain-relieving agent. Exemplary topical pain-relieving agents include lidocaine, benzocaine, novocaine, diphenhydramine, and pramoxine.

Other examples of therapeutic agents include hormones (e.g., estrogen, estradiol, estriol, estropipate, testosterone, progesterone, DHEA, testosterone, or a combination thereof), contraceptive agents (e.g., impairs sperm function, thickens cervical mucus, or both), agents that enhance vasodilation (e.g., In some embodiments, an agent that enhances vasodilation is L-arginine, niacin, nicotinamide, alprostadil, a phosphodiesterase inhibitor), erectile dysfunction treatment or erectile enhancement drugs (e.g., alprostadil, glyceryl trinitrate, sildenafil, tadalafil, vardenafil, avanafil, lodenafil, mirodenafil, udenafil, zaprinast, Icariin, benzamidenafil, dasantafil), and premature ejaculation drugs (e.g., selective serotonin reuptake inhibitors including sluoxetine, paroxetine, sertraline; tricyclic antidepressants including clomipramine).

Yet another example of a therapeutic agent is a skin conditioner or emollient.

In certain embodiments, the topical, isotonic compositions of the present disclosure further comprise at least one genital probiotic bacterial species or strain (e.g., belonging to the genus Lactobacillus). In certain embodiments, the probiotic bacterial species or strain is one having the ability to colonize the human vagina or penis. The adhesion of lactobacilli to the uroepithelium varies among species and strains, as shown by in vitro studies (Reid et al., J. Urol. 138:330, 1987), and may be mediated by glycoprotein and carbohydrate adhesins binding to glycolipid receptors (Boris et al., Infection and Immunity 66:1985, 1998). In some embodiments, a genital probiotic species is a species that is part of the genital microbiota (e.g., vagina or penis). In a specific embodiment, a genital probiotic species is selected from Lactobacillus acidophilus, Lactobacillus jensenii, Lactobacillus gasseri, Lactobacillus iners, Lactobacillus crispatus, Lactobacillus plantarum, Lactobacillus fermentum, Lactobacillus brevis, Lactobacillus casei, Lactobacillus delbrueckii, Lactobacillus vaginalis, Lactobacillus salivarius, Lactobacillus reuteri, and Lactobacillus rhamnos, Streptococcus, non-pathogenic Prevotella species, Bacillus, or any combination thereof.

The topical, isotonic compositions of the present disclosure may further comprise additional pharmaceutical excipients. Pharmaceutically acceptable excipients for therapeutic use are well known in the pharmaceutical art, and are described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

Compositions of the present disclosure can be formulated as a liquid, semi-solid, soap, gel, jelly, film, foam, cream, douche, ointment, lotion, spray, aerosol, suspension, emulsion, or paste. In certain embodiments, the compositions are formulated in a single-use or unit-dose format. Particularly in single-use compositions, the compositions may be free or substantially free (e.g., less than 1% w/w or less than 0.1% w/w) of preservatives.

In certain embodiments, topical, isotonic compositions of the present disclosure are integrated into a tampon, vaginal ring, cervical cup, diaphragm, condom, wipe, blanket, undergarment, or diaper. In certain embodiments, the topic, isotonic compositions are administered using a syringe, a roller ball, foam dispenser, spray bottle, aerosol dispenser, or pump dispenser.

In certain embodiments, compositions of the present disclosure are integrated into a microbiota sample collection and recovery system, such as a sterile swab or cyto-brush. In certain embodiments compositions of the present disclosure are used to facilitate the transplantation, storage, and cultivation of desirable gastrointestinal, vaginal, genital, reproductive tract, urinary tract, and/or anogenital microbiota from healthy donors for use in the treatment of microbial dysbiosis in affected recipients. For example, a vaginal rinse, using the compositions of the present disclosure may be collected from the vagina of healthy user and transferred to a subject with a dysbiotic microbiome of the anogenital and/or urogenital region, to promote beneficial microbiome transplantation. Such procedures are described in A Lev-Sagie, et al., Nature Medicine 25 (2019): 1500-1504, hereby incorporated by reference in its entirety. In another example, using the compositions of the present disclosure, a wipe may be used by a parent to collect beneficial genital microbiome species to transfer these via topical application to an infant born by Cesarean section.

In certain embodiments, compositions of the present disclosure may be used to add and facilitate the implantation process associated with tampons, reusable silicon devices such as menstrual cups for the control of natural menstrual flow in women or lochia post-partum and/or pelvic prolapse devices. Kits are also provided comprising a device for insertion into the vagina for control of natural menstrual flow such as a tampon and/or reusable silicon device, wherein the device is packaged in a composition of the present disclosure. For example, the compositions may be formulated with a pH-balanced gel for use in a tampon lubricant. These gels may contain a lactic acid buffer designed to maintain vaginal pH between 3.8 and 4.2 for 5 to 10 hours or 6 to 9 hours or 8 hours.

In certain embodiments, the composition is tailored to the individual recipient and donor profile to maximize the efficacy of the microbiota transplantation for each individual (e.g., the composition may be formulated for the production of specific bacteria in a user's microbiome). In certain embodiments, the composition would sustain beneficial microorganisms such as Lactobacillus acidophilus, Lactobacillus jensenii, Lactobacillus gasseri, Lactobacillus crispatus, Lactobacillus plantarum, Lactobacillus fermentum, Lactobacillus brevis, Lactobacillus casei, Lactobacillus delbrueckii, Lactobacillus vaginalis, Lactobacillus salivarius, Lactobacillus reuteri, Lactobacillus rhamnosus, and combinations thereof. In addition, compositions may also sustain other bacterial species of the Leptotrichia, Leuconostoc, Pediococcus, Akkermansia, Streptococcus, Faecalibacterium and Weissella genera during the time interval between collection and implantation. In certain embodiments, compositions of the present disclosure may inhibit and/or minimize the growth of organisms that are or can become pathogenic including the species Lactobacillus iners, or species from the genus Prevotella, Eggerthellia, Gardnerella, Atopobium, Megasphaera, Mobiluncus, Mageeibacillus, Gemella, Veillonella Sneathia Clostridium, and combinations thereof. The growth inhibition and/or minimization may occur in microbial transplantation sample storage media.

The compositions may be formulated with thermoresponsive polymers which undergo a phase change exhibiting a sol-gel transition in response to body temperature, pH, and specific ions present in physiological environments. Such thermoresponsive polymers may prolong the residence time of the composition in the urogenital and/or anogenital region (e.g., vagina). Exemplary thermoresponsive polymers include poloxamers, styrene-butadiene block copolymer, polymethylacrylate, polybutylmethacrylate, plasticized polyvinylchloride, plasticized nylon, plasticized polyethyleneterephthalate, polyethylene, polyacrylonitrile, polytrifluoro chloroethylene, poly-4,4′-isopropylenediphenylene carbonate, polyethylenevinyl esters, polyvinylchloride-diethyl fumarate, and combinations thereof.

In various implementations, the compositions further comprise one or more sustained release polymers. Suitable sustained release polymers include sodium alginate (e.g., with barium chloride), barium chloride, poly-l-lysine, polyvinylamine, protamine sulfate, and combinations thereof.

Vaginal rings or pessaries are devices inserted in the vagina to achieve controlled release of the active composition. Medicated vaginal rings may be fabricated from Silastic® 382 medical grade elastomer. The most commonly used polymers for vaginal ring are ploy (dimethylsiloxane) or silicon devices, or ethylene vinyl acetate. Additionally, biodegradable polymers, such as polycaprolactone have been used to prepare these devices. These are generally polymeric rings in which the drug (e.g., borneol, pharmaceutically acceptable salts thereof, or prodrugs of any of the foregoing such as bornyl acetate, prebiotic oligosaccharide, metal co-factor) and/or composition is homogeneously dispersed. In order to achieve constant release, two types of system are typically developed for vaginal rings: sandwich and reservoir type. In the sandwich type, a narrow layer of the active components is placed between non-medicated central core and non-medicated outer band. In reservoir type, central core having the active components is encapsulated with drug-free polymer layer (e.g., hypromellose).

The compositions and/or active ingredients (e.g., borneol, pharmaceutically acceptable salts thereof, or prodrugs of any of the foregoing such as bornyl acetate, prebiotic oligosaccharide, metal co-factor) may also be formulated in vaginal suppositories, ovules, or pessaries. In these embodiments, the suppository, ovule, or pessary may be formulated with mixture of synthetic triglycerides (e.g. Witepsol H-15), hypromellose, polyethylene glycol polymers (e.g. PEG 1000, 4000), Sorbitan monostearate (Span 60), PEG-35 Castor Oil, PEG 400, PEG 3350, cocoa butter, polyethylenimine, mixtures of mono/di and triglycerides (e.g. Suppocire, Ovucire), Agar, Propylene glycol, Hypromellose, gelatin, glycerin, cocoa butter, bees wax, Polyoxyl 40 stearate, Polyvinyl alcohol, hydroxyethyl methacrylate, polyacrylic acid, polyoxyethylene, and combinations thereof.

In some embodiments, the composition may be formulated as an emulsion. The emulsion may be, for example, a water-in-oil, oil-in-water, silicone-in-water, water-in-silicone, polyol-in-oil, oil-in-polyol, glycerin-in-oil, oil-in-glycerin, silicone-in-glycerin, glycerin-in-silicone, silicone-in-polyol, or polyol-in-silicone emulsion. The topical isotonic formulations of the present disclosure may be used as the aqueous phase (e.g., the internal phase, the discontinuous phase) of an emulsion. In an emulsion, will be understood that the weight percentage of components used herein (e.g., borneol, pharmaceutically acceptable salts thereof, or prodrugs of any of the foregoing such as bornyl acetate, prebiotic oligosaccharide, metal co-factor) refers to the weight percentage of the composition (and not the weight percentage of the phase itself). The nonaqueous phase may comprise saturated (lauric, myristic and capric acid) and unsaturated fatty acids (oleic acid, linoleic acid and linolenic acid), surfactants, and co-surfactants. Exemplary components in emulsions include sorbitan oleate (Span80), Sorbitane trioleate (Span85), polyethylene glycol sorbitan monolaurate (Tween80), Soybean oil, squalene, lecithin, oleic acid, medium chain triglyceride, Polyoxyl 40 Hydrogenated Castor Oil, Polyoxyl 35 castor oil, Glycerol, Propylene glycol, and combinations thereof.

In certain implementations, the emulsion is a Pickering emulsion. Typically, Pickering emulsions are emulsions stabilized by solid particles including nanocellulose, graphene oxide, carbon nanotube, carbon lamp black, laponite, montmorillonite, silica nanoparticles, calcium carbonate, titanium dioxide, magnetic particles, polymer particles, and combinations thereof.

In some embodiments, the compositions or active ingredients (e.g., borneol, pharmaceutically acceptable salts thereof, or prodrugs of any of the foregoing such as bornyl acetate, prebiotic oligosaccharide, metal co-factor) are formulated as a tablet. For example, the composition may be in tablet form such as chitosan and/or sodium alginate based bio-adhesive tablets. In certain embodiments, the table further comprises a mucoadhesive. The compositions or active ingredients (e.g., borneol, pharmaceutically acceptable salts thereof, or prodrugs of any of the foregoing such as bornyl acetate, prebiotic oligosaccharide, metal co-factor) may be formulated as vaginal bioadhesive tablets. Vaginal bioadhesive tablets may comprise hydroxypropyl cellulose, polyacrylic acid, Carbopol-934, and combinations thereof.

In some embodiments, the compositions or active ingredients (e.g., borneol, pharmaceutically acceptable salts thereof, or prodrugs of any of the foregoing such as bornyl acetate, prebiotic oligosaccharide, metal co-factor) are formulated as liposomes (e.g., vaginal liposomes). Vaginal liposomes include lecithin-based liposomes which may incorporate bio-adhesive carbopol hydrogels. In some embodiments, the composition comprises a thermo-sensitive gel of poloxamers 407 and 188 and are in the form of active ingredients (e.g., borneol, pharmaceutically acceptable salts thereof, or prodrugs of any of the foregoing such as bornyl acetate, prebiotic oligosaccharide, metal co-factor) loaded cationic liposomes.

The compositions (e.g., gels), applicators, and/or kits of the present disclosure may be subjected to ozone sterilization. Wherein embodiments of the present disclosure may be exposed to ozone gas due to its oxidative potential. The possibility to alter different process parameters (e.g., time of exposure, gas concentration, humidity) allows the sterilization protocol to be adapted to different types of material.

The compositions (e.g., gels), applicators, and/or kits of the present disclosure may be subjected to one or more sterilization techniques such as steam heat sterilization, filtration sterilization, gamma irradiation sterilization, e-beam sterilization, or combinations thereof. These sterilization techniques may exclude, kill, or reduce the number of microorganisms present in the final composition. Various permutations of these sterilization techniques (e.g., time of exposure, pore size, radiation dose, temperature) may allow the sterilization protocol to be adapted to different materials in the compositions, devices, systems, applicators, kits, and sensors described herein.

Exemplary formulations according to the present disclosure are provided in Tables 1-31 and throughout the examples. In some embodiments, the compositions may comprise at least the pH adjusting agent, a buffer, bornyl acetate, a metallic cofactor and a prebiotic oligosaccharide. The remaining components may be optionally present in the amounts indicated.

TABLE 1

| Ingredient Name | % by wt. |
|---|---|
| Water | q.s. |
| pH adjusting agent (e.g., NaOH, | Adjusted to acidic pH |
| lactic acid) | (e.g., pH from 3.8 to |
| | 6.8, from 4 to 4.5, |
| | from 3.8 to 6, pH |
| | from 4 to 5, pH from |
| | 5 to 6) |
| Preservative (e.g., sodium benzoate, | Less than 0.1 |
| sodium dehydroacetate) | |
| Buffering agent (e.g., monosodium | 0.001-10 |
| phosphate, disodium phosphate, | |
| gluconolactone, combinations | |
| thereof) | |
| Tonicity agent (e.g., sodium | 0.001-10 |
| chloride) | |
| Prebiotic oligosaccharide | 0.01-1 |
| Essential oil comprising bornyl | 0.01-10 |
| acetate (e.g., Abies sibirica | |
| (siberian fir)) | |
| Rheology modifier (e.g., | 0.05-20 |
| hypromellose, hydroxypropyl | |
| guar gum, combinations thereof) | |
| Humectant (e.g., arabinogalactan) | Less than 0.1 |
| Biofilm inhibiting agent (e.g., | 0.01-0.5 |
| Mentha spicata extract) | |
| Flavonoid (e.g., Citrus reticulata) | 0.001-0.1 |
| Sesquiterpene alcohol | 0.001-0.1 |
| Metallic co-factor (e.g., Manganese | 0.001-1 |
| chloride) | |

TABLE 2
Formula A

| Ingredient Name | % by wt. |
|---|---|
| Purified water, USP | q.s. |
| Sodium hydroxide | To pH 4.5 |
| Monosodium phosphate | 0.483 |
| Sodium chloride | 0.100 |
| Lactulose | 0.050 |
| Abies sibirica (siberian fir) | 0.020 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate). For example, in this embodiment the composition may comprise 0.003% (w/w) MnCl₂ (MW = 125.9 g/mol) as added from MnCl₂•4H₂O (MW = 197.9 g/mol). Therefore, 0.00472 g hydrate were added per 100 g of formulation.

TABLE 3
Formula B

| Ingredient Name | % by wt. |
|---|---|
| Purified water, USP | q.s. |
| Sodium hydroxide | To pH 6.8 |
| Monosodium phosphate | 0.483 |
| Sodium chloride | 0.100 |
| Lactulose | 0.050 |
| Abies sibirica (siberian fir) | 0.020 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 4
Formula C

| Ingredient Name | % by wt. |
|---|---|
| Purified water, USP | q.s. |
| Sodium hydroxide | To pH 4.5 |
| Disodium phosphate | 0.531 |
| Lactic acid | 0.500 |
| Sodium chloride | 0.0500 |
| Lactulose | 0.0500 |
| Abies sibirica (siberian fir) | 0.0200 |
| Hypromellose | 0.6000 |
| Gluconolactone | 0.5600 |
| Hydroxypropyl guar gum | 0.4000 |
| Arabinogalactan | 0.2000 |
| Mentha spicata | 0.0100 |
| Citrus paradisi | 0.0100 |
| Sodium benzoate | 0.1900 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 5
Formula D

| Ingredient Name | % by wt. |
|---|---|
| Purified water, USP | q.s. |
| Sodium hydroxide | To pH 6.8 |
| Disodium phosphate | 0.531 |
| Lactic acid | 0.500 |
| Sodium chloride | 0.0500 |
| Lactulose | 0.0500 |
| Abies sibirica (siberian fir) | 0.0200 |
| Hypromellose | 0.6000 |
| Gluconolactone | 0.5600 |
| Hydroxypropyl guar gum | 0.4000 |
| Arabinogalactan | 0.2000 |
| Mentha spicata | 0.0100 |
| Citrus paradisi | 0.0100 |
| Sodium benzoate | 0.1900 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 6
Formula E

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Bornyl acetate | 0.010 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Sodium benzoate | 0.125 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 7
Formula F

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Bornyl acetate | 0.010 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Lactobacillus Ferment | 1.750 |
| Sodium dehydroacetate | 0.056 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 8
Formula G

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Bornyl acetate | 0.010 |
| Hypromellose | 0.600 |
| Gluconolactone | 0.250 |
| Hydroxypropyl guar gum | 0.400 |
| Arabinogalactan | 0.050 |
| Mentha spicata | 0.015 |
| Neroli oil | 0.010 |
| Lactobacillus Ferment | 2.000 |
| Sodium dehydroacetate | 0.075 |
| Manganese chloride* | 0.003 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 9
Formula H

| Ingredient Name | % by wt. |
|---|---|
| Purified water, USP | q.s. |
| Sodium hydroxide | To pH 4.5 |
| Monosodium phosphate | 0.240 |
| Lactic acid | 0.500 |
| Sodium chloride | 0.300 |
| Lactulose | 0.050 |
| Abies sibrica | 0.020 |
| Hypromellose | 0.600 |
| Gluconolactone | 0.500 |
| Hydroxypropyl guar gum | 0.400 |
| Arabinogalactan | 0.050 |
| Mentha spicata | 0.010 |
| Neroli oil | 0.010 |
| Manganese chloride* | 0.005 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 10
Lubricant, pH 4.5, osmolality 150 mOsm/kg

| Ingredient Name | % by wt. |
|---|---|
| Purified water, USP | 96.1422 |
| Disodium phosphate | 0.39825 |
| Lactic Acid | 0.1425 |
| Lactulose | 0.05 |
| Abies sibirica (siberian fir) | 0.02 |
| hydroxyethyl cellulose | 0.8 |
| Oleuropein | 0.02 |
| gluconolactone | 1.00 |
| hydroxypropyl guar gum | 0.4 |
| Arabinogalactan | 1.00 |
| Mentha spicata | 0.0075 |
| Citrus reticulata | 0.0075 |
| Juniperus communis | 0.0075 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride (II) tetrahydrate). For example, in this embodiment the composition may comprise 0.0046% (w/w) MnCl₂ (MW = 125.9 g/mol) as added from MnCl₂•4H₂O (MW = 197.9 g/mol). Therefore, 0.00723 g hydrate were added per 100 g of formulation.

TABLE 11
Foaming Gel, pH 4.5, osmolality 120 mOsm/kg

| Ingredient Name | % by wt. |
|---|---|
| Deionized Water, USP | 93.840 |
| Manganese chloride* | 0.008 |
| Lactulose | 0.05 |
| Abies sibirica (siberian fir) | 0.02 |
| Cetyl Hydroxyethylcellulose | 0.5 |
| Oleuropein | 0.02 |
| sodium cocoyl glutamate | 2.00 |
| Sodium Lauroamphoacetate | 2.00 |
| Arabinogalactan | 1.00 |
| disodium phosphate | 0.39825 |
| Lactic Acid | 0.1425 |
| Mentha spicata | 0.008 |
| Citrus reticulata | 0.005 |
| Juniperus communis | 0.008 |

*The indicated weight percentage of metallic co-factor (i.e., magnesium chloride) may be achieved by adding the appropriate amount of a hydrate of the metallic salt (e.g., magnesium chloride (II) tetrahydrate).
† The indicated weight percent of Cocamidopropyl betaine is the weight percent from a 36% solutions of cocamidopropyl betaine may be added to each solution.

TABLE 12
Wipe Formula, pH 6.5 (infant 0-12 mo)
or pH 5 (child > 1 yr), 180 mOsm/kg

| Ingredient Name | % by wt. |
|---|---|
| Deionized Water, USP | 94.861 |
| Lactulose | 0.100 |
| Abies sibirica (siberian fir) | 0.020 |
| Cetyl Hydroxyethylcellulose | 0.500 |
| Oleuropein | 0.020 |
| Cocamidopropyl Betaine^{†} | 2.100 |
| Arabinogalactan | 0.900 |
| Citric Acid | 0.098 |
| disodium phosphate | 0.386 |
| Citrus reticulata | 0.010 |
| Manganese chloride* | 0.0046 |
| sodium benzoate | 0.3000 |
| gluconolactone | 0.7000 |

*The indicated weight percentage of metallic co-factor (i.e., magnesium chloride) may be achieved by adding the appropriate amount of a hydrate of the metallic salt (e.g., magnesium chloride (II) tetrahydrate).
^{†}The indicated weight percent of Cocamidopropyl betaine is the weight percent from a 36% solution of cocamidopropyl betaine may be added to each solution.

TABLE 13
Lubricant, pH 6.8, osmolality 340 mOsm/kg

| Ingredient Name | % by wt. |
|---|---|
| Purified Water, USP | 95.67 |
| disodium phosphate | 0.70 |
| Lactic Acid | 0.18 |
| Lactulose | 0.03 |
| sodium chloride | 0.15 |
| Raffinose | 0.02 |
| Abies alba | 0.01 |
| hydroxyethyl cellulose | 0.80 |
| Oleuropein | 0.02 |
| Hydantoin | 1.00 |
| hydroxypropyl guar gum | 0.40 |
| Arabinogalactan | 1.00 |
| Monarda Fistulosa | 0.01 |
| Citrus reticulata | 0.01 |
| Rosmarinus officinalis | 0.00 |
| Manganese chloride* | 0.01 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 14
Douche formulation

| Ingredient Name | % by wt. |
|---|---|
| Deionized Water, USP | 98.019 |
| Monosodium Phosphate, anhydrous | 0.483 |
| Sodium Chloride | 0,100 |
| Lactulose | 0.050 |
| Abies sibirica (Siberian fir) | 0.020 |
| Hypromellose | 0.600 |
| Gluconolactone | 0.250 |
| Hydroxypropyl guar gum | 0.400 |
| Arabinogalactan | 0.050 |
| Mentha spicata (spearmint) | 0.015 |
| Neroli oil | 0.010 |
| Manganese chloride | 0.003 |
| Lactic Acid 10% | Titrate to pH |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 15
Douche formulation, pH 4.5

| Ingredient Name | % by wt. |
|---|---|
| Deionized Water, USP | 99.061 |
| Monosodium Phosphate, anhydrous | 0.241 |
| Sodium Chloride | 0.200 |
| Lactulose | 0.050 |
| Abies sibirica (Siberian fir) | 0.020 |
| hypromellose | 0.400 |
| Mentha spicata (spearmint) | 0.015 |
| Neroli Oil | 0.010 |
| Manganese chloride* | 0.003 |
| Lactic acid 10% | Titrate To pH |
| | 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 16
Foaming Gel formulation, pH 4.6

| Ingredient Name | % by wt. |
|---|---|
| Deionized Water, USP | 97.3375 |
| Sodium chloride | 0.1000 |
| Lactulose | 0.0500 |
| Rosemary essential oil | 0.0500 |
| Abies sibirica (Siberian fir) | 0.0100 |
| Hypromellose | 0.3000 |
| Green tea polyphenols | 0.0200 |
| Arabinogalactan | 0.1000 |
| Mentha spicata (spearmint) | 0.0200 |
| Neroli Oil | 0.0100 |
| Manganese chloride* | 0.0025 |
| Sodium Cocyl Isethionate | 1.0000 |
| Cocamidopropyl Betaine† | 1.0000 |

*The indicated weight percentage of metallic co-factor (i.e., magnesium chloride) may be achieved by adding the appropriate amount of a hydrate of the metallic salt (e.g., magnesium chloride (II) tetrahydrate).
†The indicated weight percent of Cocamidopropyl betaine is the weight percent from a 36% solution of cocamidopropyl betaine may be added to each solution.

TABLE 17
Lubricant pH 4.5

| Ingredient Name | % by wt. |
|---|---|
| Purified Water, USP | 95.997 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lactobacillus Ferment | 2.000 |
| Sodium dehydroacetate | 0.075 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 18
Lubricant pH 4.5

| Ingredient Name | % by wt. |
|---|---|
| Purified Water, USP | 95.072 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lactobacillus Ferment | 3.000 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 19
Lubricant pH 4.0

| Ingredient Name | % by wt. |
|---|---|
| Purified Water, USP | 97.922 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Sodium dehydroacetate | 0.150 |
| NaOH | QS pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 20
Lubricant pH 4.0

| Ingredient Name | % by wt. |
|---|---|
| Purified Water, USP | 97.772 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Glyceryl caprylate | 0.300 |
| NaOH | QS pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 21
Lubricant pH 4.5

| Ingredient Name | % by wt. |
|---|---|
| Purified Water, USP | 97.672 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Euxyl K 903 | 0.400 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 22
Lubricant pH 4.5

| Ingredient Name | % by wt. |
|---|---|
| Purified Water, USP | 97.872 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lincoserve BDHA | 0.200 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 23
Lubricant pH 4.5

| Ingredient Name | % by wt. |
|---|---|
| Purified Water, USP | 97.072 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lincoserve WpH-LO | 1.000 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 24
Lubricant pH 4.0

| Ingredient Name | % by wt. |
|---|---|
| Purified Water, USP | 97.572 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Linatural-Ultra 3 | 0.500 |
| NaOH | QS pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 25
Lubricant pH 4.5

| Ingredient Name | % by wt. |
|---|---|
| Purified Water, USP | 97.572 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Linatural MBS-1 | 0.500 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 26
Lubricant pH 4.5

| Ingredient Name | % by wt. |
|---|---|
| Purified Water, USP | 97.572 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Lincoserve SSB | 0.500 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 27
Lubricant pH 4.5

| Ingredient Name | % by wt. |
|---|---|
| Purified Water, USP | 97.947 |
| monosodium phosphate | 0.240 |
| sodium chloride | 0.300 |
| Lactulose | 0.050 |
| Bornyl Acetate | 0.010 |
| hypromellose | 0.600 |
| gluconolactone | 0.250 |
| hydroxypropyl guar gum | 0.400 |
| Arabinogalactan | 0.050 |
| Mentha spicata (spearmint) | 0.015 |
| Neroli oil | 0.010 |
| Manganese chloride* | 0.003 |
| Sodium Benzoate | 0.125 |
| NaOH | QS pH 4.5 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 28
Lubricant/Tampon Insertion Gel (TIG) Formulatior

| | No | |
|---|---|---|
| | Preservative | |
| Ingredient Name | % By Wt. | Preservative |
| Purified Water, Usp | 97.465 | 97.315 |
| Monosodium Phosphate | 0.240 | 0.240 |
| Sodium Chloride | 0.450 | 0.450 |
| Lactulose | 0.050 | 0.050 |
| Abies Sibirica | 0.020 | 0.020 |
| Hypromellose | 1.200 | 1.200 |
| Gluconolactone | 0.500 | 0.500 |
| Arabinogalactan | 0.050 | 0.050 |
| Mentha Spicata | 0.010 | 0.010 |
| Citrus Aurantium Var. Amara | 0.010 | 0.010 |
| Manganese Chloride* | 0.005 | 0.005 |
| Sodium Dehydroacetate | | 0.1500 |
| Sodium Hydroxide | Titrate To | Titrate To |
| | pH 4.0 | pH 4.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 29
Cleanser 1

| Ingredient Name | % by Wt. |
|---|---|
| Deionized Water | 88.8125 |
| Sodium Chloride | 0.6000 |
| Lactulose | 0.0500 |
| Rosmarinus Officinalis | 0.0250 |
| Abies Sibirica | 0.0100 |
| Poloxamer 188 | 10.0000 |
| Arabinogalactan | 0.1000 |
| Gluconolactone | 0.2500 |
| Mentha Spicata | 0.0200 |
| Citrus Aurantium Var. Amara | 0.0100 |
| Manganese Chloride* | 0.0025 |
| Sodium Dehydroacetate | 0.12 |
| Lactic Acid | Titrate To |
| | pH 5.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 30
Cleanser 2

| Ingredient Name | % by Wt. |
|---|---|
| Deionized Water | 95.6325 |
| Sodium Chloride | 0.7500 |
| Raffinose | 0.0500 |
| Rosmarinus officinalis | 0.0250 |
| Abies sibirica | 0.0100 |
| Phenethyl Alcohol | 0.1500 |
| Propanediol | 0.7500 |
| Pentylene Glycol | 0.6000 |
| Arabinogalactan | 1.0000 |
| Mentha Spicata | 0.0200 |
| Citrus Aurantium Var. Amara | 0.0100 |
| Manganese Chloride* | 0.0025 |
| Sodium Cocoyl Glutamate (37.5% Active) | 0.50 |
| Sodium Lauroamphoacetate (30%) | 0.50 |
| Lactic Acid | Titrate To |
| | pH 5.0 |

*The indicated weight percentage of metallic co-factor (i.e., magnesium chloride) may be achieved by adding the appropriate amount of a hydrate of the metallic salt (e.g., magnesium chloride (II) tetrahydrate).

TABLE 31
Formulation 061819C, pH 6.8

| Ingredient Name | % by wt. |
|---|---|
| Purified Water, USP | 96.8684 |
| Hypromellose | 0.6 |
| Disodium phosphate, anhydrous | 0.531 |
| Gluconolactone | 0.5 |
| Lactic acid | 0.5 |
| Hydroxypropyl guar gum | 0.4 |
| Sodium hydroxide | 0.206 |
| Arabinogalactan | 0.2 |
| Sodium chloride | 0.1 |
| Lactulose | 0.05 |
| Abies sibirica | 0.02 |
| Citrus paradisi | 0.01 |
| Mentha spicata (spearmint) | 0.01 |
| Manganese chloride* | 0.0046 |

*The indicated weight percentage of metallic co-factor (i.e., magnesium chloride) may be achieved by adding the appropriate amount of a hydrate of the metallic salt (e.g., magnesium chloride (II) tetrahydrate).

It will be understood that components may have multiple purposes as those described herein. For example, citric acid may be considered a pH modifying agent and a buffering agent. Many of the components described in these tables are optional.

### In Vitro and In Vivo Activity

### (1) Evaluating Genital Microbiota

To assess whether topical, isotonic compositions of the present disclosure are harmful to the genital microbiota, testing of normal genital microbiota may be performed using methods known in the art.

The effect of any composition disclosed herein on genital microbiota (e.g., Lactobacillus species, Acinetobacter species) may be determined by measuring minimal inhibitory concentration, the lowest concentration which prevents visible growth of a microorganism after overnight culture, or minimal microbicidal concentration, the lowest concentration required to reduce the viability of a culture by, for example, more than 99% or more than 99.9% or more than 99.99%.

In some embodiments, the effect of any composition disclosed herein on genital microbiota (e.g., Acinetobacter species) may be determined by DNA extraction and 16S rRNA gene sequencing and operational taxonomic units (OTUs) assignment and community states (CST) of vaginal microbiome can be defined using Jensen-Shannon divergence and Ward linkage hierarchical clustering following administration, as disclosed in X Hong, et al., PeerJ 7 (2019): e8131, which is hereby incorporated by reference in its entirety.

Biogenic amines, amino acids, and metabolites are also biomarkers of BV and dysbiosis, as they facilitate the outgrowth of BV-associated taxa by (i) amino-acid decarboxylation that consumes intracellular hydrogen ions and change bacterial acid resistance; (ii) limiting the growth and resistance of host immunology to urogenital pathogens; and iii) being correlated with numerous host disease states, including STDs, cancer and dementias. To measure, samples may be eluted from swabs in sterile molecular water and subjected to both liquid and gas chromatography mass spectrometry.

The presence of pathogenic bacteria such as Acinetobacter species can also be assessed by measuring the concentration of endotoxins, lipopolysaccharides (LPS), and quantity of pathogenic bacteria in washes obtained from subjects following the use of compositions of the present disclosure (see, e.g., Aroutcheva et al., Anaerobe 14:256, 2008). High LPS concentrations create a toxic vaginal environment causing epithelial and gamete (e.g., sperm) damage. Even very low levels of LPS (e.g., 0.1μ/mg) rapidly impact sperm function (see, Li et al., Tohoku Journal of Experimental Medicine 238:105, 2016, incorporated herein by reference in its entirety). Since adenosine triphosphate (ATP) is present in all microorganisms, such as bacteria, measuring the presence of ATP indirectly indicates the presence of bacteria. Many ATP detection methods utilize bioluminescence to determine the presence of ATP by first exposing a sample to an ATP-releasing agent (e.g., lysis buffer) and an ATP-activated light-producing substrate and enzyme (e.g., luciferin and luciferase). The amount of ATP may be quantified by measuring the light produced by the enzymatic reaction which is in relative light units (RLU). The light may be detected at an appropriate wavelength depending on the specific ATP assay (e.g., 525 nm to 640 nm). A variety of luciferase assays and their luminogenic reagents and conditions are known in the art, and may be readily used or adapted for use herein. See, e.g. Eed, H R et al., “Bioluminescence-Sensing Assay for Microbial Growth Recognition,” Journal of Sensors, vol. 2016, Article ID 1492467, 5 pages, 2016. https://doi.org/10.1155/2016/1492467; Mempin, et al. Release of extracellular ATP by bacteria during growth. BMC Microbiol 13, 301 (2013) doi: 10.1186/1471-2180-13-301Fan F, Wood K V, “Bioluminescent assays for high-throughput screening”. Assay Drug Dev Technol. 5 (1): 127-36, 2007; Meisenheimer, et al., “Luminogenic enzyme substrates: The basis for a new paradigm in assay design,” Promega Notes 100:22-26, 2008 at http://www.promega.com/pnotes/100/16620-22/1662-0 22.pdf.

Other methods that may be used to assess changes in genital microbiota following exposure to a topical, isotonic composition of this disclosure include performing 16S rRNA gene sequencing, shotgun metagenomic gene sequencing, microbial/host metabolomic profiling, and 3^{rd} generation sequencing utilizing nanopore DNA sequencing (see, e.g., Romero et al., Microbiome 2:4, 2014; Macklaim et al., Microb. Ecol. Health Dis. 26:27799, 2015), species specific quantitative PCR (Zozaya-Hinchliffe et al., J. Clin. Microbiol. 48:1812, 2010, each of which is incorporated herein by reference in its entirety), and phylogenetic microarrays (Paliy and Agans, FEMS Microbiol. Ecol. 79:2, 2012; Chen et al., Nat Commun. 8:875, 2017, each of which is incorporated herein by reference in its entirety) using bacterial samples obtained from subjects (e.g., washes, swabs).

In certain embodiments, the compositions of the present disclosure do not reduce normal genital microbiota population (e.g., L. crispatus, L. gassseri, L. jensenii, L. acidophilus, or any combination thereof) by more than 35%, 30%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% when exposed to a concentration or amount of the composition that is to be used in vivo on a subject.

In certain embodiments, the compositions of the present invention do not interfere with acid-producing bacterial growth and functional medium acidification of fluids or solutions of the genital microbiome (e.g., Lactobacillus spp) (see, Boskey et al., Infect Immun. 67:5170, 1999, incorporated herein by reference in its entirety). For example, the composition may not interfere during in vivo application to a subject as described herein.

In certain embodiments, the compositions of the present disclosure do not promote the growth of pathogenic bacteria of the anogenital region by more than 1%, 2%, 3%, 4%, 5%, 6%, 7%, 8%, 9%, 10%, 11%, 12%, 13%, 14%, 15%, 16%, 17%, 18%, 19%, 20%, 25%, 30%, or 35%. In certain embodiments, the pathogenic bacteria are selected from pathogenic strains of Prevotella, Eggerthellia, Gardnerella, Atopobium, Megasphaera, Mageeibacillus, Mobiluncus, Bacteroides, Peptostreptococcus, Acinetobacter, Fusobacterium, Veillonella, Porphyromonas and Eubacterium.

### (2) Evaluating Genital Irritation, Inflammation, and Cell Death

Irritation, inflammation, or cell death of genital tissues, such as the vaginal mucosa or penile foreskin cells, can be assessed in vitro or in vivo using human or animal vaginal-ectocervical, urethral, or foreskin tissue explants; vaginal, cervical or vulvar cell monolayers; penile epithelium or urethral epithelium; skin cell monolayers; slug mucosal irritation assays; or other equivalent methods. In order to predict the safety of the composition of the disclosure, instead of using animal testing (e.g., rabbit vaginal irritation (RVI) test, guinea pig maximization test (GPMT), acute systematic toxicity tests using rabbits or mice), in vitro methods based on human reconstructed tissue models may be performed. In one embodiment, a non-animal vaginal irritation method may be used to evaluate irritation of the isotonic composition disclosed herein. Briefly, a non-clinical assessment model (NAM), such as commercially available human reconstructed tissue models (e.g., EpiVaginal™ (MatTek Corporation; Ashland, MA); human vaginal epithelium (HVE) (SkinEthic (Lyon, France)) may be used for testing. See, e.g., Costin, G-E., et al. “Qualification of a non-animal vaginal irritation method admitted as nonclinical assessment model (NAM) in the Incubator Phase of the United States Food and Drug Administration (US FDA) Medical Devices Development Tool (MDDT).” Toxicology in Vitro 62 (2020): 104680, which is incorporated herein by reference in its entirety and particularly with respect to the NAMs.

For example, the slug mucosal irritation assay (SMI) is a sensitive system to detect even mild mucosal irritation potency (Adriaens et al., Sex. Transm. Dis. 35:512, 2008, incorporated herein by reference in its entirety). The SMI assay uses slugs (Arion lusitanicus) as the test organism. The body wall of slugs consists of a mucosal surface comprising mucus secreting cells covering a sub epithelial connective tissue. Slugs that are placed on an irritant substance will actively produce mucus as a protective mechanism from noxious agents. Additionally, tissue damage of the slug's surface results in the release of proteins and enzymes. The protein concentration in the collected samples is determined with a protein quantitation kit. A composition of the present disclosure is considered non-irritating if it does not cause an increased production of mucus, or an increased release of proteins and enzymes as compared to a negative control.

Human, organotypic vaginal-ectocervical tissue models produced from normal human-derived vaginal-ectocervical epithelial cells may also be used to assess irritation of topically applied products (Ayehunie et al., Toxicology 279:130, 2011; Ayehunie et al., 2007, Toxicology 279:130, 2007; Trifonova et al., Antimicrob. Agents Chemother. 50:4005, 2006; Fichorova et al., mBio 2: e00168, 2011, each of which is incorporated herein by reference in its entirety). Release of markers of cell damage (e.g., increase in CD4, IL-1B, CXCL8, CCL2, CCL21, EMP1; decrease in BPI) and production of inflammatory mediators, such as IL-1, IL-8, TLR4, may be used as markers of irritation and pro-inflammation (see, also, Fichorova et al., Toxicol. Appl. Pharmacol. 285:198, 2015; Doncel et al., J. Acquir. Immune Defic. Syndr. 37 (Suppl. 3): S174, 2004; Fichorova et al., Biol. Reprod. 71:761, 2004; Moench et al., BMC Infect. Dis. 10:331, 2010, each of which is incorporated herein by reference in its entirety). Biomarkers of epithelial integrity and immune function have been validated in multiple clinical studies of vaginal product safety (Mauck et al., AIDS Res. Hum. Retroviruses 29:1475, 2013; Fichorova et al., mBio, 6: e00221, 2015; Fichorova et al., Cytokine 55:134, 2011; Mauck et al., J. Acquir. Immune Defic. Syndr. 49:243, 2008; Morrison et al., J. Acquir. Immune Defic. Syndr. 66:109, 2014; Schwartz et al., Contraception 74:133, 2006; Keller et al., J. Antimicr. Chemother. 51:1099, 2003, each of which is incorporated herein by reference in its entirety). A composition of the present disclosure may be considered non-irritating and non-inflammatory if it does not cause more than a 25% release of markers of cell damage or expression of pro-inflammatory mediators above that caused by a negative control (e.g., synthetic TLR2/6 ligand). In some embodiments, the compositions of the present disclosure may be considered non-irritating and non-inflammatory to genital skin and/or mucosa if application of the composition to the specific region does not increase inflammasome and/or cytokine production (e.g., by more than 1%, more than 5%, more than 10%, more than 15%, more than 20%, more than 25%) as compared to control. For example, a control reference level may be the level of the indicated biomarker expressed as an average of the level of the biomarker from samples taken from a control population of healthy subjects. In some embodiments, a control reference level may be the level of the indicated biomarker expressed as an average of the level of the biomarker measured from a subject given a control formulation. Suitable samples or references for determining reference levels include healthy cells. In some embodiments, the reference to determine the reference level of an indicated biomarker may be a derived from the subject, a healthy subject, or a population of subjects.

Healthy mucin and mucus, and mucin-regulating enzyme production (glycosidases), from epithelial cells can be determined following exposure of genital tissue or fluid to compositions used for cleaning and lubricating the anogenital region. Current urogenital and/or anogenital products can damage the natural mucus protection barrier of the surface of genital skin or mucosa of the vagina, penis and urethra, through altering mucin production and enhancing mucin-degradation. In particular, mucin degradation can occur following exposure to certain pathogenic bacteria or to certain ingredients (e.g. carbomer and oils) commonly found in products used in the genital region. Mucin quality from the vagina can be determined following exposure to a composition of the present disclosure by testing CVF samples collected using a SoftCup or similar menstrual cup device covering the base of the cervix and performing ELISA assay (enzyme linked immunosorbent assay) to measure mucins and ELLA assay (enzyme linked lectin assay) to measure carbohydrate structures as described in Moncla et al. (PLOS One 11: e0158687, 2016, incorporated herein by reference in its entirety). Glycosidase assays can be performed to measure enzyme specific activity as described in Moncla, incorporated herein by reference in its entirety). A composition of the present disclosure is generally considered non-harmful to genital mucin and mucus if it does not inhibit mucus viscosity or production (e.g., by more than 1%, 5%, 10%, 15%, 20%, 25%, 30%, or 35%), or increase production of glycosidases (e.g., by more than 1%, 5%, 10%, 15%, 20%, 25%, 30%, or 35%) (see, e.g., Moncla). Compositions of the present disclosure may also be tested for their effects in vaginal infection susceptibility models, such as a mouse genital herpes transmission model (see, e.g., Moench et al., BMC Infect. Dis. 10:331, 2010). Increased susceptibility to infection transmission may be caused by damage to vaginal epithelial cells.

Effects of topical compositions on tissue viability using tissue models (e.g. human explants or cell monolayers) may also be assessed using the MTT colorimetric assay technique. The MTT assay is a colorimetric assay for assessing cell metabolic activity. NAD (P) H-dependent cellular oxidoreductase enzymes may, under defined conditions, reflect the number of viable cells present. These enzymes are capable of reducing the tetrazolium dye MTT 3-(4,5-dimethylthiazol-2-yl)-2,5-diphenyltetrazolium bromide to its insoluble formazan, which has a purple color. The MTT assay may be used to measure a composition's cytotoxicity or effect on cell viability (see, e.g., Ayehunie et al., 2011).

In addition, oxidative stress and antioxidant potential of the tissues can be determined by common methods, such as a TBARS assay to evaluate the impact of various embodiments on tissue health. Because reactive oxygen species (ROS) have extremely short half-lives, they are difficult to measure directly. Instead, several products of the damage produced by oxidative stress, such as thiobarbituric acid reactive substances (TBARS), can be measured. TBARS are formed as a byproduct of lipid peroxidation (i.e., as degradation products of fat), which can be detected by the TBARS assay using thiobarbituric acid as a reagent.

The in vivo rabbit vaginal irritation (RVI) model may also be used to assess the irritation and inflammatory characteristics of a formulation (see, e.g., Eckstein et al., J. Reprod. Fertil. 20:85, 1969). This model is based on macroscopic observations of erythema, edema and ulceration and histopathologic analysis of the tissues collected after exposure of the animals to the test materials. A non-irritating and safe composition of this disclosure would show no negative macroscopic or histopathologic effects as compared to a control vehicle. An expanded RVI model having a quantitative nuclease protection assay (qNPA) to quantify mRNA levels of 25 genes representing leukocyte differentiation markers, toll-like receptors (TLR), cytokines, chemokines, epithelial repair, microbicidal and vascular markers has also been described (see, e.g., Fichorova et al., Toxicol. Appl. Pharmacol. 285:198, 2015).

Sensitization tests may be used evaluate the potential of a composition of the present disclosure to cause a sensitizing effect or allergenic reaction in a subject over an extended period of exposure. Exemplary tests include Guinea pig tests, such as the Magnusson-Kligman guinea pig maximization test (J. Invest. Dermatol. 52:268, 1969), the occluded patch test of Buehler (Arch. Dermatol. 91:171, 1965), and the open epicutaneous test (see, e.g., Kero et al., Contact Dermatitis 6:341, 1980). A murine local lymph node assay (LLNA) is another method for the identification of skin sensitizing chemicals. In contrast to guinea pig tests, this assay relies on measurement of events induced during the induction phase of skin sensitization, specifically lymphocyte proliferation in the draining lymph nodes which is a hallmark of a skin sensitization response, rather than the elicitation phase (see, e.g., Kimber et al., Contact Dermatitis 21:215, 1989; Basketter et al., Food Chem. Toxicol. 34:985, 1996). The human repeat-insult patch test (HRIPT) may be performed as a confirmatory test in the safety evaluation of skin sensitizers. Sensitization is a process by which humans develop increased allergic responses to a substance over time through repeated exposure to that substance. It is different from irritation because it involves an immune response. Skin sensitization reactions are usually characterized by erythema coupled with one or more of various dermal sequelae, such as edema, papules, vesicles, bullae, and/or pruritus (see, e.g., McNamee et al., Regul. Toxicol. Pharmacol. 52:24, 2008).

Irritating topical products may trigger the release of pro-inflammatory cytokines (e.g., TLR, IL-1, IL-6, IL-8, TNF-α, IFN-γ, IL-17) and inflammasomes (e.g., NLRP3 and NLRC4). Cytokines and inflammasomes can be measured using an enzyme-linked immunosorbent assay (ELISA), quantitative PCR, or other molecular assay. A product is considered non-inflammatory if it does not cause increased expression of pro-inflammatory cytokines or inflammasomes (Rabeony et al., Eur. J. Immunol. 45:2847, 2015).

In certain embodiments, the compositions of the present disclosure do not induce irritation or inflammation potential in the anogenital region subject greater than 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30% as compared to an untreated control subject, preferably as measured using the slug mucosal irritation test of Adriaens et al. (2008).

### (3) Evaluating Effect on Gametes

In certain embodiments, the compositions of the present disclosure do not impact sperm viability or function. In certain embodiments, compositions of the present disclosure are designed to mimic the vaginal environment during a woman's fertile window around ovulation and not negatively impact sperm viability or function. In some embodiments, the compositions of the present disclosure are designed to mimic the vaginal environment during a woman's non-fertile period and not impact sperm viability or function. Assays or models for assessing sperm survival and function, include for example, sperm motility assays (e.g., subjective or computer assisted), sperm viability studies, in vitro fertilization and embryo development animal models, membrane integrity of sperm, survival time in culture, cervical mucus penetration, lipid peroxidation, capacitation, zona recognition, acrosome reaction and sperm-oocyte fusion, and sperm chromatin testing (reviewed in, e.g., Vasan, Indian J. Urol. 27:41, 2011; Ochninger et al., Fertil. Steril. 102:1528, 2014; Mortimer et al., Hum. Reprod. Update 19 (Suppl 1): i1-i45; 2013, each of which is incorporated by reference in its entirety). Additional testing can include post-coital testing to evaluate sperm presence in the cervical canal, and even pregnancy outcomes in an animal model or among women in a clinical trial.

Sperm motility is one function that may be used to assess sperm function and thus fertilization potential. Motility of sperm is expressed as the total percent of motile sperm, the total percent of progressively motile sperm (swimming forward), or the speed of sperm that are progressively motile. These measurements may be made by a variety of assays. For example, either a subjective visual determination is made using a phase contrast microscope when the sperm are placed in a hemocytometer or on a microscope slide, or a computer assisted semen analyzer is used. Under phase contrast microscopy, motile and total sperm counts are made, and speed is assessed as fast, medium or slow. A more specific measurement of sperm motility is motility grade, where the motility of sperm is divided into four different grades (Cooper et al., Human Reprod. Update 16:231, 2010, incorporated by reference in its entirety). Grade A refers to sperm with progressive motility that are the strongest and swim fastest in a straight line. Grade B refers to sperm with non-linear motility; that move forward but tend to travel in a more curved or crooked motion. Grade C sperm have non-progressive motility in that they do not move forward despite tail movement. Grade D sperm are immotile. Using a computer assisted semen analyzer (such as IVOS Hamilton Thorne, Beverly, MA), the motility characteristics of individual sperm cells in a sample are objectively determined (Hum. Reprod. 13:142, 1998). Briefly, a sperm sample is placed onto a slide or chamber designed for the analyzer. The analyzer tracks individual sperm cells and determines motility and velocity of the sperm. Data may be expressed as percent of total motility, and measurements are obtained for path velocity and track speed as well. It is known that the velocity of sperm is often impacted by the viscosity of a medium and separate from the toxicology of that medium as described in J Elgeti, et al., Biophys J 99 (2010): 1018-1026, hereby incorporated by reference in its entirety.

The Human sperm survival assay is typically used in human in vitro fertilization (IVF) programs as described in C De Jonge et al., J Androl 24 (2003): 16-18, and A Hossain, et al., Adv Urol 2010 (2010): 136898, each of which is hereby incorporated by reference in its entirety. It has also been proposed as a sensitive cytotoxic assay for any topical products, as sperm show damage before other cell monolayer types. The test requires sperm suspension culture with 10% product for 24 hours and evaluation of the percent motile, progressive motility and/or total motility of sperm at the end of culture in climate-controlled settings. The test can also be replicated with bull sperm for controlling for individual sperm sample effect. In certain embodiments, the compositions of the present disclosure are considered not toxic to sperm if application to a subject does not cause a decrease in sperm survival greater than 0.5%, greater than 1%, greater than 2%, greater than 3%, greater than 4%, greater than 5%, greater than 6%, greater than 7%, greater than 8%, greater than 9%, greater than 10%, greater than 11%, greater than 12%, greater than 13%, greater than 14%, greater than 15%, or greater than 20% as compared to control.

Sperm viability can be measured using several different methods. By way of example, two of these methods are staining with membrane exclusion stains and measurement of ATP levels. Briefly, a sample of sperm is incubated with a viable dye, such as Hoechst 33258 or eosin-nigrosin stain. Cells are placed in a hemocytometer and examined microscopically. Dead sperm with disrupted membranes stain with these dyes. The number of cells that are unstained is divided by the total number of cells counted to give the percent live cells. ATP levels in a sperm sample are measured by lysing the sperm and incubating the lysate with luciferase, an enzyme obtained from fireflies, which fluoresces in the presence of ATP. The fluorescence is measured in a luminometer. The amount of fluorescence in the sample is compared to the amount of fluorescence in a standard curve allowing a determination of the number of live sperm present in the sample.

Membrane integrity of sperm may be assayed by a hypo-osmotic swell test which measures the ability of sperm to pump water or salts if exposed to non-isotonic environments. Briefly, in the hypo-osmotic swell test, sperm are suspended in a solution of 75 mM fructose and 25 mM sodium citrate, which is a hypo-osmotic (150 mOsm) solution. Sperm with intact, healthy membranes pump salt out of the cell causing the membranes to shrink as the cell grows smaller. The sperm tail curls inside this tighter membrane. Thus, sperm with curled tail are counted as live, healthy sperm with normal membranes. When compared to the total number of sperm present, a percent of functional sperm may be established.

The degree of membrane integrity may be determined by lipid peroxidation (LPO) measurements, which assess sperm membrane damage generated by free radicals released during handling. Lipid membrane peroxidation is assayed by incubating sperm with ferrous sulfate and ascorbic acid for one hour in a 37° C. water bath. Proteins are precipitated with ice-cold trichloroacetic acid. The supernatant is collected by centrifugation and reacted by boiling with thiobarbituric acid and NaOH. The resultant malondialdehyde (MDA) formation is quantified by measuring absorbance at 534 nm as compared to an MDA standard (Bell et al., J. Andrology 14:472, 1993, incorporated herein by reference in its entirety). LPO is expressed as nM MDA/108 sperm. A composition of the present disclosure has a stabilizing effect on sperm if exposure results in decreased LPO production.

The stability of chromatin DNA is assayed using the sperm chromatin structure assay (SCSA). Sperm cells are very sensitive to oxidative stress resulting in sperm chromatin (DNA) damage (Whittington et al., Int. J. Andrology 22:236, 1999; Pasqualotto et al., Fertility and Sterility 73:459, 2000; Kodama et al., Fertility and Sterility 68:519, 1997, each of which is incorporated herein by reference in its entirety). This damage can be profound in sperm cells because they contain little to no mechanisms to repair DNA damage after it occurs. The sperm chromatin assay is based on the metachromatic staining of single and double stranded DNA by acridine orange stain (Evenson et al., Human Reprod. 14:1039, 1999; Evenson et al., J. Andrology 23:25, 2002, each of which is incorporated by reference in its entirety). Excitation with an argon laser causes acridine orange intercalated in double-stranded DNA to emit a green fluorescence, whereas red fluorescence is emitted by single strand DNA. The extent of DNA denaturation in a sample is expressed as a and calculated by the formula α=red/(red+green). The endpoint measurement is DNA Fragmentation Index (DFI). A DFI of <15% DFI indicates excellent to good sperm DNA integrity. Fresh sperm samples are incubated for a period of time in the presence of a test composition, flash frozen, and subsequently assayed for DNA breakage (see, e.g., Evenson et al., J. Androl. 23:25, 2002, incorporated herein by reference in its entirety). Other DNA assays for the stability of chromatin DNA include the terminal deoxynucleotidyl transferase-mediated fluorescein-dUTP nick end labelling (TUNEL) test; COMET assay and Sperm Chromatin Dispersion as disclosed in D Evenson, Anim Reprod Sci 169 (2016): 56-75, hereby incorporated by reference in its entirety.

In vitro fertilization rates are determined by measuring the percent fertilization of oocytes in vitro in an animal model such as bovine or murine model. For example, maturing bovine oocytes are cultured in vitro in M199 medium plus 7.5% fetal calf serum and 50 μg/ml luteinizing hormone for 22 hours. Following culture for 4 hours, the sperm are chemically capacitated by adding 10 IU of heparin and incubated with bovine oocytes for 24 hours. At the end of the incubation, oocytes are stained with an aceto-orcein stain or equivalent to determine the percent oocytes fertilized. Alternatively, fertilized oocytes may be left in culture for 2 days, during which division occurs and the number of cleaving embryos (i.e., 2 or more cells) is counted.

Survival time in culture of sperm (time to loss of motility) is another convenient method of establishing sperm function. Briefly, an aliquot of sperm is placed in culture medium, such as Tyrode's medium, pH 7.4 and incubated at 37° C., 5% CO₂, in a humidified atmosphere. At timed intervals, for example every 2 hours, the percentage of motile sperm in the culture is determined by visual analysis using an inverted microscope or with a computer assisted sperm analyzer. As an endpoint, a sperm sample is considered no longer viable when less than 5% of the cells have progressive motility.

Another parameter of sperm function is the ability to of sperm to swim up into a column of cervical mucus or substitute (reviewed in Ola et al., Hum. Reprod. 18:1037-1046, 2003, incorporated by reference in its entirety). This cervical mucus penetration test can be done either in vitro or in vivo. Sperm are placed at one end of the track and the distance that sperm have penetrated into the mucus after a given time period is determined. Cervical mucus penetration studies offer valuable biocompatibility data for devices that are used for reproductive purposes. The bovine cervical mucus penetration study is an excellent in vitro assay to evaluate sperm penetration into cervical mucus. Bergman et al. (Fertility and Sterility 36:363-367, 1981, incorporated herein by reference in its entirety) found excellent correlation between sperm penetration into frozen bovine cervical mucus and fresh human cervical mucus (r=0.98) due to the similarity of human and bovine cervical mucus rheological and biophysical make-up (Bergman et al., Fertil. Steril. 36:363-367, 1981; Keel et al., Arch. Androl. 44:109-115, 2000, each of which is incorporated herein by reference in its entirety). These assays may be used to evaluate cytotoxicity of aspects of the present disclosure, such as applicators, compositions, and kits, by incubating the embodiment (e.g. applicator) in a container with a mixture of the composition (e.g. 10% composition by volume of the solution), cervical mucus and a sperm solution, in order to determine toxicity of leached products from the device on sperm penetration into cervical mucus. Toxicity of sperm penetration can also be measured by placing the compositions of the present disclosure in an applicator (e.g., an applicator as described herein), and incubating the composition with the applicator for a set time (e.g. more than 10 min, more than 20 min, more than 25 min, 30 min), where after the incubated composition is mixed with cervical mucus and sperm to form a solution. This solution may be used to evaluate subsequent sperm penetration into mucus, to determine effects of sperm exposed to the composition and/or identify the presence of any leached chemicals from the applicator into the composition. In certain embodiments, the compositions of the present disclosure are not toxic to sperm if there is not a decrease in sperm penetration of greater than 0.5%, of greater than 1%, of greater than 2%, of greater than 3%, of greater than 4%, of greater than 5%, of greater than 6%, of greater than 7%, of greater than 8%, of greater than 9%, of greater than 10%, of greater than 11%, of greater than 12%, of greater than 13%, of greater than 14%, of greater than 15% of control.

Other assays for sperm function include global proteomic or metabolomic assays, or assays providing metatranscriptomic (mRNA), or glycomic (sugars in situ) analysis of sperm (e.g., 24 hr following ejaculation). These assays may evaluate sub-cellular changes for proteins related to hyperactivation, capacitation, acrosome reaction and zona-pellucida binding (fertilization process proteins). Such assays may be conducted using liquid chromatography/mass spectroscopy (LC-MS-MS) or alternatively NMR and data matched to establish proteomics online databases.

In other aspects, microbiome (e.g., penile, genital) sequencing from a subject with V4 16s rRNA gene copies or whole genome sequencing may be performed. Microbial DNA may be extracted from swabs taken of the microbiome region to be measured using standard extraction protocols and sequenced on high through put genomic sequencing platforms such as nanopore sequencing, or next generation sequencing platforms (such as Illumina Hi seq). Using online bioinformatics pipelines standard to the industry, raw reads from the sequencing data may be curated and filtered for quality control. Finalized sequenced data can be compared to annotated reference databases in order to determine the composition of a sample's microbiome, and biological observation matrices may be employed, for example, for visualization and statistical analyses. Furthermore, correlation analysis of microbiota in the penile region or semen may be used to determine if a sample needs optimization such as being associated with known pathogenic bacteria. The status of a sample can be compared to other sperm function tests to determine if there are any pathogenic bacteria, and in particular, pathogenic bacteria that may be causing sperm infertility issues.

Alternatively, sperm penetration of mucus may be measured in vivo in women. At various times post-coitus, a sample of cervical mucus is removed and examined microscopically for the number of sperm present in the sample. In the post-coital test, improved sperm function is established if more sperm with faster velocity are seen in the mucus sample after exposure to a composition of the present disclosure versus a sample of mucus from the patient after exposure to a control lubricant.

Other assays of sperm function potential include the sperm penetration and hemizona assays. In the sperm penetration assay, the ability of sperm to penetrate into an oocyte is measured. Briefly, commercially available zona free hamster oocytes are used (EmbryoTech Laboratories, Haverhill, MA). Hamster oocytes are suitable in this assay for sperm of any species. Capacitated sperm, such as those cultured with bovine serum albumin for 18 hours, are incubated for 3 hours with the hamster oocytes. Following incubation, oocytes are stained with acetolacmoid or equivalent stain and the number of sperm penetrating each oocyte is counted microscopically. A hemizona assay measures the ability of sperm to undergo capacitation and bind to an oocyte. Briefly, in this assay, live normal sperm are incubated in media with bovine serum albumin, which triggers capacitation. Sperm are then incubated with dead oocytes which are surrounded by the zona pellucida, an acellular coating of oocytes. Capacitated sperm bind to the zona and the number of sperm binding is counted microscopically.

In certain embodiments, a composition of the present disclosure may be considered non-toxic to sperm if following exposure to a 10% solution of the composition, sperm retain at least 80%, at least 85%, at least 90%, or at least 95% motility as compared to sperm exposed to a control medium.

In certain embodiments, the topical, isotonic composition of the present disclosure: (i) is non-irritating to the urogenital and/or anogenital mucosa or skin; (ii) does not promote growth of pathogenic bacteria of the urogenital and/or anogenital region such as species from the genus Acinetobacter including Acinetobacter baumannii; (iii) does not reduce the healthy microbiota of the genital region more than 25%; (iv) does not disrupt or reduce mucin production by the urogenital and/or anogenital mucosa or skin; (v) does not cause more than a 20% increase in inflammation of the urogenital and/or anogenital mucosa and/or the skin genital tissue; (vi) does not disrupt genital fluid function; (vii) is non-toxic to sperm; (viv) has a prebiotic effect on Lactobacillus species growth found in the genital tissues; (viv) decreases bacterial, fungal, or viral infection rates by 5% or more; or any combination thereof.

The present disclosure is partially premised on compositions which are primarily focused on creating the optimal microbiotic environment for healthy growth (e.g. Lactobaccilus growth) and decreasing pathogenic growth (e.g., Acinetobacter growth) in the anogenital and/or urogenital microbiomes. In some embodiments, the compositions may be used for the treatment or prophylaxis of a disease, disorder, or condition associated with dysbiosis of the urogenital and/or anogenital region in a subject in need thereof. The method for the treatment of the treatment or prophylaxis of a disease, disorder, or condition associated with dysbiosis of the urogenital and/or anogenital region in a subject in need thereof may comprise administration of a composition of the present disclosure (e.g., a topical isotonic composition comprising bornyl acetate, a prebiotic oligosaccharide, and a metal co-factor) to the urogenital and/or anogenital region of the subject. In some embodiments, the urogenital and/or anogenital region is the penis. In some embodiments, the composition is administered topically to the penis. The method may further comprise measuring the pH of the penis (e.g., with a pH nanosensor or other method) prior to application, and administering a composition to affect the pH of the microbiome environment in order to physiologically optimize Lactobacillus dominance and minimize Acinetobacter growth. In various embodiments, the disease, disorder, or condition associated with dysbiosis of the urogenital and/or anogenital region may be cancer such as penile cancer.

In yet another aspect, the present disclosure provides a method for collecting the genital microbiome from a donor dyad member comprising topically administering an effective amount of a topical, isotonic composition of the present disclosure to the urogenital and/or anogenital region of the donor dyad member and collecting the topical, isotonic composition from the urogenital and/or anogenital region of the subject. In certain embodiments, the topical, isotonic composition is collected in a receptacle. In certain embodiments, the topical, isotonic composition is administered via a wipe, adhesive roller, a blanket, an undergarment, diaper, film, or aerosol. In some embodiments, the composition is integrated into a tampon, vaginal ring, cervical cup, diaphragm, or condom, wherein the composition will be released upon insertion into the urogenital and/or anogenital region.

In certain embodiments, the collected topical, isotonic composition is assayed for the presence of pathogenic microorganisms; cultured to identify and propagate beneficial microorganisms (e.g., Lactobacilli); or both. In certain embodiments, the beneficial microorganisms are isolated and added as a probiotic or by vaginal flora transfer to a separate, topical, isotonic composition of the present disclosure for administration to a recipient dyad member or other unrelated individual. In certain embodiments, the donor dyad member and recipient dyad member are members of a sexual dyad, e.g., a heterosexual dyad, homosexual dyad, or other sexual orientation dyad.

The individual components of the compositions described herein (e.g., prebiotic oligosaccharides, metal co-factors, bornyl acetate, essential oils comprising bornyl acetate) may be used for application of a subject in need thereof. In some embodiments, these individual components (e.g., prebiotic oligosaccharides, metal co-factors, bornyl acetate, essential oils comprising bornyl acetate) may be used for the preparation of a medicament (e.g., topical compositions, isotonic compositions, topical isotonic compositions) for the treatment of a subject in need thereof. For example, the individual components or the medicament may be administered to the subject in order to hydrate the urogenital and/or anogenital region of the subject and/or lubricate the anogenital region of the subject and/or clean the urogenital and/or anogenital region of the subject and/or decrease irritation or inflammation of the urogenital and/or anogenital region of the subject and/or enhance the genital microbiota of the subject. In certain embodiments, the individual components and compositions described herein may be used for the treatment or the prophylaxis of the dysbiosis of a subject in need thereof.

### Applicators

The present disclosure also includes applicators which may be used for administration to the urogenital and/or anogenital region of a subject (e.g., the vagina, the penis). The applicator may comprise a storage portion having an internal reservoir capable of storing one or more doses of the compositions of the present disclosure. In some embodiments, the internal reservoir may have a volume of from 1 mL to 60 mL or 1 mL to 5 mL or 5 mL to 60 mL or 10 mL to 50 mL or 15 mL to 30 mL or 20 mL to 25 mL. The internal reservoir may be in fluid communication with an application portion configured to release an amount of the composition. For example, a user may apply to a force to the storage portion causing the composition to be expelled therethrough.

The applicator may further comprise a sensor capable of measuring one or more characteristics of the surrounding environment (e.g., the penis, the vagina) including the pH. For example, the applicator may comprise a litmus or nitrazine dye which, following insertion, is capable if visually displaying pH information the environment of the urogenital and/or anogenital regions. In some embodiments, the applicator may comprise a nanosensor such as that disclosed in U.S. Pat. No. 10,436,745, hereby incorporated by reference in its entirety and particularly in relation to pH nanosensors. In some embodiments, the nanosensor is capable of measuring the pH of the surrounding environment (e.g., the anogenital and/or urogenital region). Following measurement, the nanosensor may be capable of communicating the pH measurement (e.g., with Bluetooth, radio frequency identification, USB such as USB-A, USB-B, USB-C, micro USB, lightning cable) with an external device such as a laptop, tablet, computer, server, and/or smart phone. The nanosensor may transmit the value of the measured pH or information relating to the pH. For example, the sensor may transmit a binary signal and/or a ternary signal depending on user settings. The external device may be configured to interpret, display, and track such measurements.

In some embodiments, the compositions of the present disclosure may be delivered using vaginal or topical films wherein the composition is capable of diffusing from the film into its surrounding environment. Suitable film formers include chitosan, hydroxypropyl methylcellulose and blends of these polymers (e.g., with 40% PEG 400 as plasticizer), a polymeric matrix/chitosan with carrageenan (κ-, λ-, and ι-), pectin and gellan gum, hydroxyl propylcellulose and sodium alginate as polymers and propylene glycol and polyethylene glycol-400 as plasticizers, polyvinyl alcohol, poloxamer 407 and 188, hypromellose, sodium carboxymethylcellulose, hydroxylpropylmethylcellulose, hydroxyethylcellulose and polyvinyl pyrrolidone K-90, hydroxypropyl methylcellulose and Eudragit polymers (e.g., Eudragit RL100) and propylene glycol as plasticizer, hydroxypropyl methylcellulose, polyvinyl alcohol, polyethylene oxide, glycerol, poly(2-oxazoline)/polyoxazoline polymers and combinations thereof.

The applicators (or any portion thereof such as the container and/or the applicator element) may be formed from those materials known in the art. In some embodiments, portions of the applicator or the entire applicator may be made low waste packaging materials such as biodegradable plastics. Suitable biodegradable plastics may be bio-based plastics such as polyhydroxyalkanoates (PHAs), polylactic acid (PLA), starch blends, cellulose-based plastics, lignin-based polymer composites, and combinations thereof. The biodegradable plastics may also be petroleum based such as polyglycolic acid (PGA), polybutylene succinate (PBS), polycaprolactone (PCL), poly(vinyl alcohol) (PVA, PVOH), polybutylene adipate terephthalate (PBAT), and combinations thereof. In some embodiments, portions of the applicator (e.g., the applicator element) or the entire applicator may be composed of paper and/or cardboard. In some embodiments, the paper and/or cardboard applicators or portions thereof, may be burned or disposed of after vaginal mucosal contact, thereby decreasing risky medical waste.

The applicator may be composed of paper or cardboard. Each composition may be contained in an enclosure (e.g., slim bag, vial, or pouch), which may, in some embodiments, be a small, flexible bag configured to connect and fill an applicator, such as but not limited to a paper and/or cardboard applicator. The enclosure for the composition or gel described herein, may be composed of ethylene-vinyl acetate (EVA), polyethylene, low density polyethylene (LDPE), high density polyethylene (HDPE), or any biodegradable or recyclable material, or combinations thereof. Another embodiment provides for an enclosure that contains a composition having an acidic pH (e.g., pH 3.5-pH 5, pH 3.8-5). Yet another embodiment provides for an enclosure containing a composition having a neutral or alkaline pH or a pH from 6.5 to 7.4. The applicator may have a paper and/or cardboard barrel and plunger, and a mechanical stop and/or a fill line which assists in the prevention of overfilling the applicator in an amount greater than the intended dose when a user or other person who may fill the applicator with the desired composition. The fill line to which the composition should be added to the applicator without exceeding the fill line, may indicate the amount of composition for a single use, multiple use, e.g., 4.0-gram amount, combinations thereof, or any amount recommended for administration. However, different amounts or volumes may similarly be indicated by the fill line depending on the composition (e.g., acidic pH, neutral or alkaline pH), dosage, usage, etc. The composition enclosure, in some embodiments, may be a pharmaceutical grade pouch which allows preservative-free dosing, with high biocompatibility and low-leeching of resins. In one embodiment, the compositions of the disclosure may be stored in the enclosure under stressed (accelerated) conditions for three months and yet maintain the integrity of the compositions stored therein.

### Kit

Yet other embodiments of the disclosure may provide for a kit or applicator dispensing carton described here. These kits or cartons may comprise any of the product applicators and the compositions described herein. The kit may be a self-contained carton providing product dispensing and waste containment capabilities. Advantages of the kit may include a design that accommodates a bulk supply (i.e., multi-unit supply) of applicators; discrete product dispensing and use for the user; low-waste production; containment and limited hazardous waste in community trash; and a decreased shipping volume.

In some embodiments, the kit may contain or be configured to hold a supply of applicators for treatment of one month or greater (e.g., 2, 3, 4, 5, 6, 7, 8, 9, 10, 11, 12). The applicators may be initially empty for filling by the user, or pre-filled, with compositions of the disclosure. Other embodiments may provide for a kit comprising or configured to hold 50% or greater (e.g., 60%, 70%, 90%) applicators; or 50% or fewer (e.g., 40%, 25%, 10%) applicators, where the applicators contain compositions having an acidic pH (e.g., pH 4-pH 5). Yet another embodiment may be directed to a kit comprising or configured to hold 50% or greater (e.g., 60%, 70%, 90%) applicators; or 50% or fewer (e.g., 40%, 25%, 10%) applicators, where the applicators contain compositions having a neutral or alkaline pH (e.g., pH 6.5-pH 7.4).

Other embodiments may provide a kit containing 2 or more (e.g., 4, 6, 8, 10, 12, 14, 16, 18, 20, 22, 24, 26, 28, 30, 36, 40, 48, 50, 54, 56, 60, 68, 72, 78) applicators; 372 or fewer (e.g., 144, 140, 138, 132, 130, 126, 120, 114, 110, 108, 102, 100, 96, 90, 84, 78, 72, 66, 60, 54, 48, 42, 36, 30, 24, 18, 12, 6 or fewer) applicators; applicators in a range of 2-372 (e.g., 4-364, 6-288, 12-138, 18-132, 24-126, 30-120, 42-114, 48-108, 54-102, 60-96, 66-90, 72-84); or applicators in a sufficient number for a supply of one month or more (e.g., 2, 3, 4, 5, 6, 7, 8, 9, 10, 11, 12 months), where a one month supply may contain 24 or more (e.g., 25, 26, 27, 28, 29, 30, 31) applicators. The kit may provide for a waste containment receptacle and an easy and discrete applicator dispenser(s) with or without a gel or composition unit or enclosure dispenser. Other embodiments may be directed to a kit or carton that provides all product needs for the composition and its use, without external inputs (e.g., no water, batteries, or the like would be necessary).

The kit described here may also contain instructions for use and/or a dosing schedule. The dosing schedule may be included as a sticker on the external carton surface. In other embodiments, the kit may be of any sized dimensions sufficient to hold the desired number of applicators or composition units, and a waste receptacle for holding the used and disposed of applicators or composition units. The carton may be sufficiently large to hold a 1 month to 12 months' supply of the applicators and composition(s), for example a three months' supply. With respect to the dimensions of the kit, a kit designed to contain a close-packing ensemble of components is desirable.

### EXAMPLES

### Example 1: Decrease Acinetobacter Species in Microbiota

The penile microbiota was measured from three different healthy men (N=3) following after three days of a test condition. Subjects were either 1) not administered a composition (baseline), 2) administered Astroglide® topically to the penile region, or 3) administered a composition of the present disclosure comprising a metallic cofactor, a prebiotic oligosaccharide, and bornyl acetate (BioMa) topically to the penile region. Microbiota were measured for the relative abundance of pathogenic Acinetobacter species. Microbiota were also measured for the relative abundance for Veillonella species, a genital microbe associated with fertility.

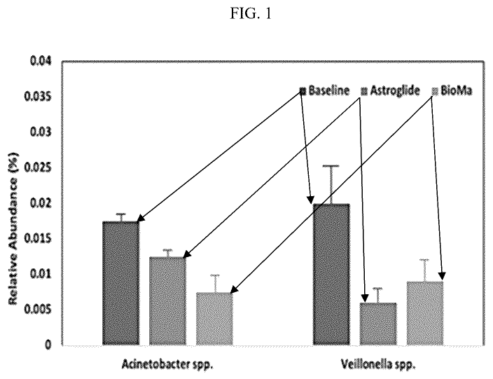

FIG. 1 provides the results of these measurements. As can be seen, administration of the compounds of the present disclosure correlates with a reduction in Acinetobacter abundance following topical application. Without wishing to be bound by theory, the compositions of the present disclosure may act in a multifunctional manner in order to mitigate Acinetobacter dominance prior to its becoming pathogenic. This multifunctional mechanism May 1) reduce available energy sources for Acinetobacter growth, 2) reduce the ability of Acinetobacter to shear the O-linked glycoalyx mucins that protect against pathogenic growth, 3) prevent pH and metabolomic sway, 4) reverse the reductive oxidant environment that promotes growth of Acinetobacter in the microbome, 5) alter iron metabolism and siderophore function to limit pathogenic growth.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet and Request are incorporated herein by reference, in their entirety. Aspects of the embodiments can be modified, if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of decreasing Acinetobacter growth in the microbiome in the genital region of a subject in need thereof comprising application of a pharmaceutical composition having
(a) a metallic co-factor;
(b) a prebiotic oligosaccharide; and
(c) borneol or a prodrug thereof.

2. The method according to claim 1, wherein said composition is buffered with a buffer system comprising gluconolactone.

3. The method according to claim 1, wherein said composition has an acidic pH.

4. The method according to claim 1, wherein the administration promotes beneficial bacterial growth.

5. The method according to claim 1, wherein the application has minimal effect to the male gametes in any genital fluids secreted from the subject or the sexual partner of the subject.

6. The method according to claim 5, wherein the male gametes have minimal change in their motility and/or concentration and/or vitality and/or morphology and/or oxidation-reduction potential and/or sperm DNA fragmentation and/or sperm mitochondrial membrane potential and/or survival and/or sub-cellular alterations.

7. The method according to claim 1, wherein said composition has a pH of from 3.5-5.

8. The method according to claim 1, wherein said composition comprises an essential oil comprising bornyl acetate.

9. The method according to claim 1, wherein said composition comprises a biofilm inhibiting agent.

10. The method according to claim 1, wherein the composition comprises a preservative.

11. The method according to claim 10, wherein the preservative is an α-hydroxy acid.

12. The method according to claim 1, wherein the composition comprises less than 1% preservative by weight of the composition.

13. The method according to claim 1, wherein said composition is administered at least once daily (e.g., once daily, twice daily, three times daily).

14. The method according to claim 1, wherein said method improves a penile cancer treatment, treats or prevents penile cancer, and/or decreases urogenital infection in the subject.

15. The method according to claim 1, wherein the Acinetobacter growth is decreased in the microbiome in the population of the Acinetobacter albensis, Acinetobacter apis, Acinetobacter baumannii, Acinetobacter baylyi, Acinetobacter beijerinckii, Acinetobacter bereziniae, Acinetobacter bohemicus, Acinetobacter boissieri, Acinetobacter bouvetii, Acinetobacter brisouii, Acinetobacter calcoaceticus, Acinetobacter celticus, Acinetobacter chengduensis, Acinetobacter colistiniresistens, Acinetobacter courvalinii, Acinetobacter cumulans, Acinetobacter defluvii, Acinetobacter dispersus, Acinetobacter dijkshoorniae, Acinetobacter equi, Acinetobacter gandensis, Acinetobacter gerneri, Acinetobacter guangdongensis, Acinetobacter guerrae, Acinetobacter guillouiae, Acinetobacter gyllenbergii, Acinetobacter haemolyticus, Acinetobacter harbinensis, Acinetobacter indicus, Acinetobacter junii, Acinetobacter kookii, Acinetobacter lactucae, Acinetobacter lanii, Acinetobacter larvae, Acinetobacter lwoffii, Acinetobacter modestus, Acinetobacter nectaris, Acinetobacter nosocomialis, Acinetobacter oryzae, Acinetobacter parvus, Acinetobacter pakistanensis, Acinetobacter populi, Acinetobacter portensis, Acinetobacter proteolyticus, Acinetobacter pittii, Acinetobacter piscicola, Acinetobacter pragensis, Acinetobacter proteolyticus, Acinetobacter pseudolwoffii, Acinetobacter pullicarnis, Acinetobacter pullorum, Acinetobacter puyangensis, Acinetobacter qingfengensis, Acinetobacter radioresistens, Acinetobacter rudis, Acinetobacter schindleri, Acinetobacter seifertii, Acinetobacter shaoyimingii, Acinetobacter soli, Acinetobacter stercoris, Acinetobacter tandoii, t Acinetobacter tjernbergiae, Acinetobacter towneri, Acinetobacter ursingii, Acinetobacter variabilis, Acinetobacter venetianus, Acinetobacter vivianii, Acinetobacter wanghuae, Acinetobacter wuhouensis, and combinations thereof present in the microbiome.

16. The method according to claim 1, wherein the Acinetobacter growth is decreased in the microbiome in the population of the Acinetobacter baumannii in the microbiome.
